(19) United States
(12) Patent Application Publication
(10) Pub. No.: US 2006/0178560 A1
(43) Pub. Date: Aug. 10, 2006

(54) Endoluminal tool deployment system

(75) Inventors: Vahid Saadat, Saratoga, CA (US); Chris A. Rothe, San Jose, CA (US); Richard C. Ewers, Fullerton, CA (US); Tracy D. Maahs, Rancho Santa Margarita, CA (US); Kenneth J. Michlitsch, Livermore, CA (US)

Correspondence Address: LEVINE BAGADE HAN LLP, 2483 EAST BAYSHORE ROAD, SUITE 100, PALO ALTO, CA, 94303, US

(73) Assignee: USGI Medical, Inc., San Clemente, CA (US)

(21) Appl. No.: 11/400,120

(22) Filed: Apr. 7, 2006

Related U.S. Application Data
(63) Continuation of application No. 10/797,485, filed on Mar. 9, 2004.
Application No. 10/797,485 is a continuation-in-part of application No. 10/735,030, filed on Dec. 12, 2003.
Application No. 10/797,485 is a continuation-in-part of application No. 10/458,060, filed on Jun. 9, 2003.
Application No. 10/458,060 is a continuation-in-part of application No. 10/346,709, filed on Jan. 15, 2003.
(60) Provisional application No. 60/471,893, filed on May 19, 2003.

(51) Int. Cl. A61B 1/008

(52) U.S. Cl. 600/114; 600/141; 600/146

(57) ABSTRACT

Systems, devices and methods for endoscopic procedures are provided involving accessing and manipulating tissues beyond the capabilities of traditional endoscopic instruments. Embodiments of the systems include an elongated main body which has one or more independently shape-lockable sections and a variety of instruments which are either built in to the main body or advanceable through lumens which extend through the main body. Such instruments may include scopes, suction instruments, aspiration instruments, tool arms, plicators, needles, graspers, and cutters, to name a few. The ability to steer and shapelock specific sections of the main body enables access to target locations which are typically challenging to reach and provides a stabilized platform to perform a desired procedure at the target location.

### CROSS-REFERENCE TO RELATED APPLICATIONS

This application is a continuation of U.S. patent application Ser. No. 10/797,485, filed Mar. 9, 2004, which is a continuation-in-part of U.S. patent application Ser. No. 10/735,030, filed Dec. 12, 2003 and a continuation-in-part of U.S. patent application Ser. No. 10/458,060, filed Jun. 9, 2003, which is a continuation-in-part of U.S. patent application Ser. No. 10/346,709, filed Jan. 15, 2003, and also claims the benefit of prior Provisional Application No. 60/471,893, filed May 19, 2003, the full disclosures of which are hereby incorporated by reference.

### STATEMENT AS TO RIGHTS TO INVENTIONS MADE UNDER FEDERALLY SPONSORED RESEARCH OR DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK.

Not applicable.

### BACKGROUND OF THE INVENTION

The present invention relates generally to medical devices, systems and methods. More particularly, the present invention relates to devices, systems and methods for use in endoscopic or laparoscopic procedures.

Endoscopy is a form of minimally invasive procedure wherein the interior of the body is accessed and visualized through an orifice in the body, such as the esophagus or rectum. Such access allows a surgeon or physician to view and/or treat internal portions of the orifice or internal tissues or organs which are accessible through the orifice. These procedures may be for diagnostic purposes, such as visual inspection or the removal of a tissue sample for biopsy, or the procedure may be used for treatment purposes, such as the removal of a polyp or tumor or the restructuring of tissue. While these procedures can be done using regular open surgery, endoscopy usually involves less pain, less risk, less scarring, and faster recovery of the patient.

Endoscopy is typically performed with the use of an endoscope, a small circular tube containing optical components. Traditional endoscopes comprise a small diameter “snake-like” insertion tube having a distal end which is inserted into the orifice to the desired internal location. Fiber optics extend through the insertion tube and terminate at the distal end to allow axial viewing from the distal end. Images of the internal location near the distal end of the endoscope are transmitted to a video monitor for the physician to view. A control handle allows the endoscopist to control the direction of the scope and in some cases, permits the actuation of air, water and suction utilities that may be required for the endoscopy procedure.

Since endoscopes may be used to perform a treatment at an internal location, some endoscopes are equipped with a lumen through which a surgical instrument or tool may be passed. Generally, the lumen extends through the length of the insertion tube to the distal end so that the end effector of the inserted instrument protrudes from the distal end in the axial direction. Thus, the instrument is directed in parallel to the fiber optics so that the end effector is positioned along the line of view.

Such endoscopes have a number of constraints which limit their usefulness in performing diagnostic and surgical procedures. To begin, surgical instruments and tools are inserted axially through a working lumen in the endoscope. Also, most of these endoscopes only allow axial and rotational movement of the tool beyond the distal end. This helps to maintain positioning of the tool within the field of view of the endoscope which is also directed axially. However, this limits the variety and complexity of procedures that may be performed. For example, procedures that involve tissue approximation pose great difficulty since only one portion of tissue may be grasped at a time and lateral, rather than axial, movement may be required. Although steering of an axially inserted tool may be possible near the distal end, such steering typically positions the end effector of the tool out of the field of view of the axially directed scope.

A similar minimally invasive procedure that overcomes some of these constraints is laparoscopy. In laparoscopy, the interior of the body is accessed and visualized through a small incision. When accessing the abdomen, the incision is usually made in the navel. Laparoscopy was initially used by gynecologists to diagnose and treat conditions relating to the female reproductive organs: uterus, fallopian tubes, and ovaries. It is now used for a wider range of procedures, including operations that in the past required open surgery, such as removal of the appendix (appendectomy) and gallbladder removal (cholecystectomy). Laparoscopy is performed with a device that allows the surgeon or physician to view and/or treat internal tissues or organs which are accessible through the incision. This device is the same or similar to an endoscope, sometimes referred to as a laparoscope. The device comprises a small diameter insertion tube having a distal end which is inserted into the incision to the desired internal location. Fiber optics extend through the insertion tube and terminate at the distal end to allow axial viewing from the distal end. Images of the internal location near the distal end are transmitted to a video monitor for the physician to view. Sometimes, access through an incision creates a shorter, straighter and more direct access path than through an orifice. Therefore, some laparoscopes may have a shorter and stiffer insertion tube than some endoscopes.

Although laparoscopes suffer from many of the same limitations as endoscopes, laparoscopy allows additional surgical instruments and tools to be inserted through separate incisions to perform procedures. Proper location of the incisions can allow instruments to be positioned in various directions. Therefore, movement and viewing is not limited to the axis of the laparoscope and simultaneous viewing of the tissues and the instruments may be more readily achieved during the procedure. However, these additional benefits are achieved at the cost of increased invasiveness. Access paths must be created for the instruments with the use of trocars requiring general anesthesia, risk of complications and infection, and increased overall recovery time for the access paths to heal. In addition, access may be difficult or contraindicated in some patients, particularly in the morbidly obese.

Thus, it would be desired to provide improved methods, devices and systems to perform minimally invasive procedures. Particularly, it would be desirable to provide methods, devices and systems which would provide the benefits of endoscopy, such as lower invasiveness and access to deeply internal locations, with the benefits of laparoscopy, such as the use of multiple instruments with movement and viewing along various axes. The devices and systems would be reliable, convenient and easy to use with improved outcomes for patients due to reduction in invasiveness and therefore risk, cost and recovery time. At least some of these objectives will be met by the invention described hereinafter.

In addition, it would be desired to provide improved methods, devices and systems which would provide improved passage and manipulation through endovascular passageways. Typical endoscopes have a length in the range of 130 to 190 cm and may be used to traverse a variety of tortuous paths within the body. For example, endoscopes may be used to access the lower gastrointestinal tract from entry through the anus, sometimes reaching as far as the cecum at the distal end of the colon. The upper gastrointestinal tract may be accessed through the esophagus to the stomach and the upper regions of the small intestine. Achieving access to any of these regions, particularly through the colon, involves tedious manipulation of the endoscope. Much of this manipulation involves torqueing of the endoscope. However, once a substantial length of the endoscope has passed into the body, torqueing becomes increasingly difficult. In addition, accessing such regions usually takes place through minimally supported lumens, such as the colon, which do not provide resistive strength or through open cavities, such as the stomach, which do not provide particular pathways for the endoscope. This also limits the use of endoscopic access to desired treatment locations.

Thus, it would be desired to provide improved methods, devices and systems to access desired treatment locations. Particularly, methods, devices and systems which would improve the ability to access desired treatment locations minimally invasively, particularly endoscopically or laparoscopically. The devices and systems would be reliable, convenient and easy to use with improved outcomes for patients due to reduction in invasiveness and therefore risk, cost and recovery time. At least some of these objectives will be met by the invention described hereinafter.

### BRIEF SUMMARY OF THE INVENTION

The present invention provides systems, devices and methods for endoscopic procedures involving accessing and manipulating tissues beyond the capabilities of traditional endoscopic instruments. Embodiments of the systems include an elongated main body which has one or more independently steerable and/or shape-lockable sections and a variety of instruments that are either built in to the main body or advanceable through lumens that extend through the main body. Such instruments may include scopes, suction instruments, aspiration instruments, tool arms, plicators, needles, graspers, and cutters, to name a few. The ability to steer and shape-lock specific sections of the main body enables access to target locations which are typically challenging to reach and provides a stabilized platform to perform a desired procedure at the target location.

In a first aspect of the present invention, a system is provided which includes an elongated main body having a proximal end and a distal end terminating in a distal tip. In some embodiments, the main body includes one or more of section A, section B and section C. Section A refers to a deflectable and/or steerable portion which may be advanced through supported or unsupported anatomy. Section B refers to a portion which is capable of retroflexion. Optionally, this section is laterally stabilized and deflectable in a single plane to facilitate steering within open cavities. Section C refers to a steerable portion, optionally steerable within any axial plane in a 360 degree circumference around the shaft. When section C is the most distal section, such steerability allows movement of the distal tip in a variety of directions. The main body may be comprised of any combination of sections A, B, and C and may include such sections in any arrangement. Likewise, the main body may be comprised of any subset of sections A, B, an C, such as section A and section C, or simply section C. Further, additional sections may be present other than sections A, B, and C.

The sections of the main body may have any suitable construction, and steering and locking may be achieved by any suitable mechanisms. In some embodiments, the one or more sections of the main body are comprised of a multiplicity of nested links or nestable elements. The elements are disposed so that a distal surface of one element coacts with a proximal surface of an adjacent element. Each of the nestable elements includes one or more pullwire lumens through which pullwires pass. The pullwires are used to hold the elements in nesting alignment and to optionally provide steering and locking.

In some embodiments, the one or more sections of the main body are comprised of bump links. Bump links allow steering of a section to a predetermined arrangement, such as a continuous radius of curvature during retroflexion. In other embodiments, one or more sections of the main body are comprised of pin links. Pin links provide lateral deflection while maintaining axial rigidity.

Pinned nested links are another type of link which may be used in the construction of the main body. Pinned nested links allow freedom of rotation of the links for steering but maintain alignment in particular locations for torque transmission. In some embodiments, the links have a slot and pin arrangement wherein a slot in a first link accepts a pin on an adjacent link. Each link is free to rotate in at least a plane defined by the alignment of pins and slots. When the position of such aligned pins and slots are varied along the length of the plurality of adjacent links, the links are able to rotate in various directions.

In yet other embodiments, one or more sections of the main body comprise a multiplicity of saddle links. Typically, a saddle link includes two flanges, each flange extending in the same direction on opposite sides of the saddle link. The flanges allow rotation of the links in the direction of the flanges yet limit rotation in other directions. Thus, saddle links may be used in sections that are desired to be steered in two directions, such as in a wagging motion.

In additional embodiments, one or more sections of the main body comprise rattlesnake links. Typically, a rattlesnake link includes two flanges, each flange extending in the same direction on opposite sides of the rattlesnake link. However, each link also includes a top edge which follows a curvature that is opposite to the bottom edge. Since the top edge of each link inversely mimics its bottom edge, the links are stacked in an alternating fashion, with each link offset 90 degrees from an adjacent link. Since the position of the flanges alternate with each link, the main body may be steered in four directions while providing torque control.

In a second aspect of the present invention, a system is provided which includes an elongated main body having a side opening. The side opening leads to an internal lumen within the main body so that an instrument may be passed through this internal lumen exiting the main body through the side opening rather than through an opening at the distal tip. When a scope is passed through the side opening, the distal tip of the main body may be visualized from an angle or from a distance which may be more desirable for a particular procedure than visualization directly from the distal tip itself.

In a third aspect of the present invention, a system is provided which includes an elongated main body having a handle that provides a variety of functions including but not limited to controlling tension in the pullwires. Tension control includes applying tension to various pullwires to steer specific portions of the main body or applying a proximal force to one or more of the pullwires to lock specific portions of the main body in a desired shape. Tension control may be provided with a variety of mechanisms, including pulleys, spools, knobs, ratchets, and gears, to name a few. In some embodiments, separate steering and locking mechanisms are present to control each section of the main body.

In a fourth aspect of the present invention, a system is provided which includes an elongated main body having a bite block connection. Bite blocks are used to assist in retaining the main body inside the mouth of the patient so as to prevent bite damage. Thus, bite blocks may be useful when the main body is used to access the upper gastrointestinal tract and/or stomach through the esophagus.

In a fifth aspect of the present invention, a system is provided which includes an elongated main body having suction capabilities. In some embodiments, the main body has a lumen through which evacuation pressure may be applied to create suction. Suction may be useful in a variety of procedures, such as gastrointestinal cleaning of fluid, biomatter or blood, or for degassing, to name a few. Further, suction may be used in plication procedures. The main body may include a suction cap near its distal end into which tissue is drawn and plicated by suction and then secured or maintained by sutures delivered by advancement of an instrument, such as a needle, through the main body. Sutures may optionally be used in combination with anchors and further with pledgets to increase the surface area of the anchors. In some embodiments, the suction cap includes a grasping feature which facilitates grasping and positioning of the suction cap at various locations to be treated.

Methods of using the apparatus, devices and systems of the present invention are also provided. Other objects and advantages of the present invention will become apparent from the detailed description to follow, together with the accompanying drawings.

### BRIEF DESCRIPTION OF THE DRAWINGS

FIG. 1 illustrates an embodiment of a system of the present invention.

FIG. 2 illustrates the system of FIG. 1 in an assembled arrangement.

FIG. 2A depicts the cross-section of the system of FIG. 2, and FIG. 2B depicts an alternative cross-section.

FIGS. 3A-3D, 4-6 illustrate possible movements of the steerable distal ends of the tool arms.

FIGS. 7A-7B illustrate the use of an embodiment of the system to perform a mucosectomy.

FIGS. 8A-8C illustrate a two-sectioned embodiment of the main body in a variety of positions.

FIGS. 8D-8E illustrate a three-sectioned embodiment of the main body in a variety of positions.

FIGS. 8F-8H illustrate the embodiment of FIGS. 8D-8E approaching the gastroesophageal junction.

FIG. 9A shows an embodiment of the shaft of the main body comprised of a multiplicity of nestable elements, and FIG. 9B provides an exploded view of these elements.

FIGS. 9C-9E provide cross-sectional views of various nestable elements.

FIG. 10A provides an exploded view of nestable elements having a pullwire extending through their centers, and FIG. 10B provides a cross-sectional view one of the nestable elements.

FIG. 10C illustrates the nestable elements of FIG. 10A with the inclusion of liners, and FIG. 10D provides a cross-sectional view of one of the nestable elements.

FIGS. 11A-11G and FIG. 12 illustrate embodiments of bump links.

FIGS. 13A-13F illustrate embodiments of pin links.

FIGS. 14A-14D illustrate embodiments of pinned nested links.

FIGS. 15A-15E and FIGS. 16A-16E illustrate embodiments of saddle links.

FIGS. 17A-17F illustrate embodiments of rattlesnake links.

FIG. 18 illustrates an embodiment of the main body having a side opening.

FIG. 18A illustrates a scope protruding from the side opening of the main body of FIG. 18.

FIG. 18B illustrates a tool arm protruding from the side opening of the main body of FIG. 18.

FIGS. 19A-C illustrate tensioning mechanisms for applying tension to pullwires.

FIG. 19D illustrates a handle for use in shape locking the main body of the present invention.

FIG. 20A illustrates a bite block for use with the main body of the present invention.

FIG. 20B illustrates an embodiment of the main body having a bite block.

FIG. 21 illustrates an embodiment of the main body having a tubing for applying suction passing therethrough.

FIG. 22A illustrates suction drawn through a main lumen of the tubing of FIG. 21.

FIG. 22B illustrates suction drawn around a scope passed through the main lumen of the tubing of FIG. 21.

FIG. 23 illustrates an embodiment of a suction cap joined with or attached to the tubing for applying suction.

FIGS. 24A-24B illustrate suction and piercing of tissue drawn into the suction cap wherein a needle is passed through a scope within the suction cap.

FIGS. 25A-25B illustrate an embodiment of a suction plication system wherein a scope and instrument guide are provided independently within the main body terminating within the suction cap.

FIGS. 26A-26G illustrate an exemplary method of plicating tissue using apparatus of the present invention.

FIGS. 27-28 illustrate exemplary methods of resecting a lesion using apparatus of the present invention.

FIG. 29 illustrates a suction cap including an exit port through which a scope is able to pass.

FIG. 30 illustrates a suction cap disposed at the end of a suction tube separate from the scope.

FIG. 31 illustrates a suction cap having a grasping feature.

FIGS. 32-33 illustrate examples of positioning the suction cap with the use of a grasper.

FIGS. 34A-34G illustrate an exemplary method of plicating tissue using apparatus of the present invention.

### DETAILED DESCRIPTION OF THE INVENTION

I. Overview

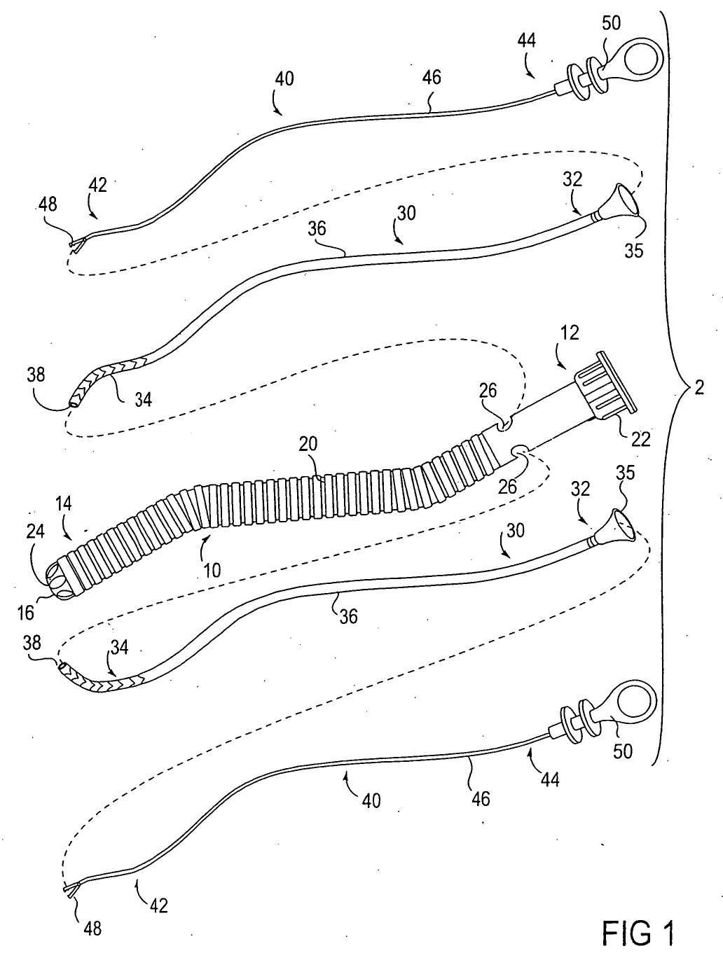

An embodiment of a system 2 of the present invention is illustrated in FIG. 1. The system 2 includes an elongated main body 10 having a proximal end 12 and a distal end 14 terminating in a distal tip 16. The main body 10 is used to access an internal target location within a patient's body. Typically, the distal end 14 is passed through a body orifice and one or more naturally occurring body lumens to the target location, such as in endoscopy, while the proximal end 12 remains outside of the body. Therefore, the main body 10 has a deflectable and/or steerable shaft 20, either due to choice of material or design of the shaft 20 to include links, hinges, coils or other similar structures to allow deflection. Thus, FIG. 1 illustrates the main body 10 in a deflected position wherein the body 10 includes curvatures. Such deflection and/or steering may be useful in traversing body lumens to the target location and is achievable by manipulation of a handle 22 near the proximal end 12. It may be appreciated, however, that the system 2 may be used in laparoscopic procedures wherein such deflection and/or steering may be less utilized for placement of the main body 10. In either case, rigidization of some or all the shaft 20 may be desired, for example to provide a stable visualization platform. Therefore, portions of the shaft 20 of the main body 10 are lockable to maintain a desired shape and provide rigidity, either due to choice of material or design of the shaft 20 to include locking mechanisms, as will be described in later sections.

The main body 10 may have a single lumen therethrough for a variety of uses, such as for passage of one or more instruments or for the passage of air or fluid, such as for aspiration or suction. Similarly, the main body 10 may have more than one lumen passing therethrough, each lumen used for a different function. In preferred embodiments, the main body 10 has an outer diameter of about 5-25 mm, more preferably approximately 14-18 mm. In some embodiments, a single lumen therethrough has an inner diameter of approximately 10-20 mm, preferably about 12-14 mm. In some embodiments having more than one lumen therethrough, the lumens are sized to fit within an inner diameter of approximately 10-15 mm.

In some embodiments, the main body 10 includes at least one instrument or tool lumen, such as an arm guide lumen 26, which extends over or through at least a distal section of the main body 10, typically along the majority of the length of the body 10 as shown. Here in FIG. 1, two arm guide lumens 26 are shown, each extending from a position along the shaft 20 near the proximal end 12 to the distal tip 16. In addition, the main body 10 includes a scope lumen 24 which extends through the shaft 20 to the distal tip 16.

In some embodiments, the system 2 also includes at least one tool arm 30, two are shown in FIG. 1, each arm 30 of which is insertable through a separate arm guide lumen 26 as indicated by dashed line. Each tool arm 30 has a proximal end 32, a distal end 34 and a shaft 36 therebetween. The distal end 34 optionally is steerable, such as by manipulation of adjacent links as schematically indicated. Such steerability may be controlled by a steering cuff 35 which is part of the proximal end 32. The shaft 36 is typically flexible or deflectable to allow deflection of the surrounding main body shaft 20. Each tool arm 30 additionally includes a tool deployment lumen 38 therethrough.

In this embodiment, the system 2 also includes at least one tool 40, two are shown in FIG. 1. Each tool 40 includes a distal end 42, a proximal end 44 and an elongate shaft 46 therebetween to allow passage through the tool deployment lumen 38 of the arm 30. Each tool 40 has an end effector 48 disposed at the distal end 42 and optionally a handle 50 at the proximal end 44 for manipulation of the end effector 48 from outside the body. The tool 40 is advanced so that the end effector 48 emerges from the distal end 34 of the arm 30. It may be appreciated that in other embodiments, the tool 40 may be passed through any other lumen of the main body or the tool 40 may be formed integrally with tool arm 30.

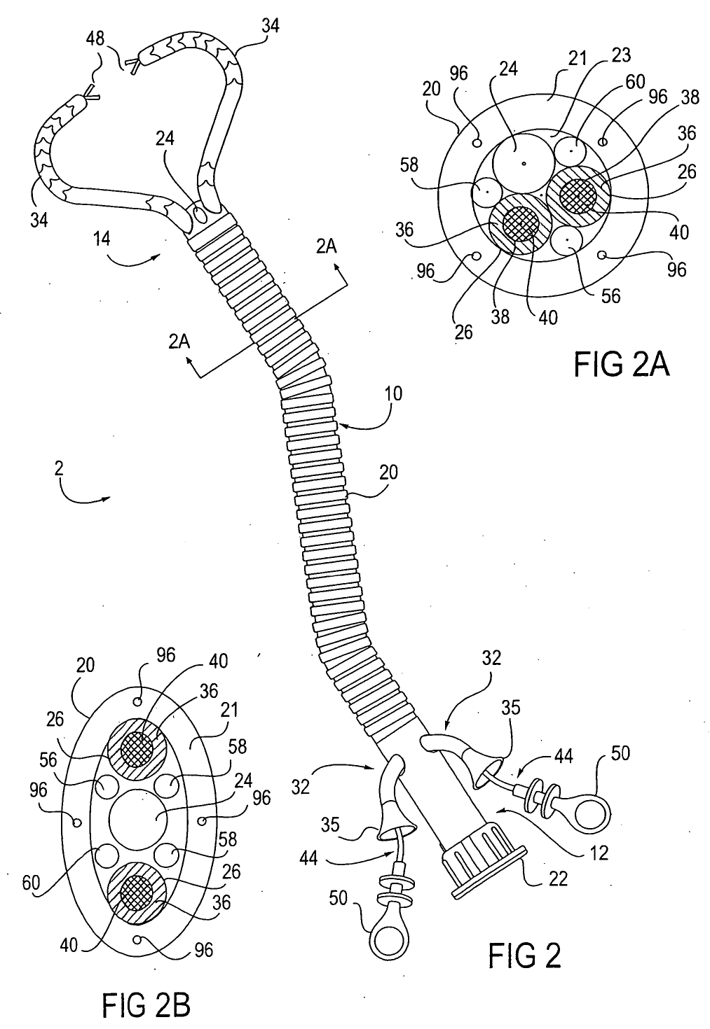

FIG. 2 illustrates the system 2 of FIG. 1 in an exemplary assembled arrangement. Here, the tool arms 30 are shown inserted through the arm guide lumens 26 of the main body shaft 20. The steerable distal ends 34 of the arms 30 protrude from the distal end 14 of the main body 10 and the proximal ends 32 of the arms 30 protrude from the proximal end 12 of the main body 10. As shown, the steering cuffs 35 are located at the proximal ends 32 of the arms 30. In addition, the tools 40 are shown inserted through the tool deployment lumens 38 so that the end effectors 48 extend beyond the steerable distal ends 34 of the arms 34. Likewise, the proximal ends 44 of the tools 40 with handles 50 are shown protruding from the steering cuffs 35. Movement of the tools 40 against the steering cuffs 35 will actuate steering of the distal ends 34 of the arms 30, as will be described in later sections.

FIG. 2A provides a cross-sectional view of system 2 of FIG. 2. Since the shaft 20 of the main body 10 has a generally cylindrical exterior in this embodiment, the cross-section of the shaft 20 has a circular shape. It may be appreciated that cylindrical shafts may alternatively have an elliptical, oval or oblong cross-section. The shaft 20 has an outer diameter in the range of about 5 to 25 mm, preferably approximately 14-18 mm. The shaft 20 has a wall 21 with a thickness in the range of about 0.5 to 5 mm, preferably about 2-3 mm, defining an inner central lumen 23. Within the wall 21 lie various pushwires or pullwires 96, e.g. wires, cables, coils, etc., hereinafter referred to as pullwires, for steering and/or locking the main body 10. The pullwires 96 may alternatively be present within the central lumen 23. As will be apparent, such pullwires 96 may be present in a variety of quantities and arrangements.

At least one arm guide lumen 26, two are shown, extend through the central lumen 23 of shaft 20. Each arm guide lumen 26 has an inner diameter in the range of about 0.5 to 10 mm, preferably about 6 mm. Positioned within the lumens 26 are the shafts 36 of the tool arms 30. And, likewise, positioned within the shafts 36 are the tools 40. FIG. 2A also illustrates the scope lumen 24 which has an inner diameter in the range of about 2 to 10 mm, preferably about 4-6 mm. In this embodiment, the two arm guide lumens 26 and the scope lumen 24 are arranged in a generally triangular pattern which is maintained to the distal tip 16, however any suitable arrangement may be used which allows viewing of the tool arms, particularly the end effectors, by the scope. For example, FIG. 2B illustrates a cross-section of an embodiment wherein the shaft 20 has an oval shape and the arm guide lumens 26 and the scope lumen 24 are generally aligned. Here, the scope lumen 24 is disposed between the arm guide lumens 26 to facilitate viewing of the tool arms 30. Also illustrated in FIGS. 2A and 2B are additional lumens which may be used for various needs. For example, an irrigation/suction lumen 60, an insufflation lumen 56 and an auxiliary lumen 58 may be present, each having an inner diameter in the range of about 0.5 to 5 mm, preferably about 2-4 mm. The auxiliary lumen 58 may be utilized for a variety of uses, such as insertion of additional tools, such as a macerator, a grasping tool, a cutting tool or a light source, to name a few, for use in conjunction with the end effectors present at the distal ends of the arms 30 or the distal ends of the tools 40 inserted through the arms 30.

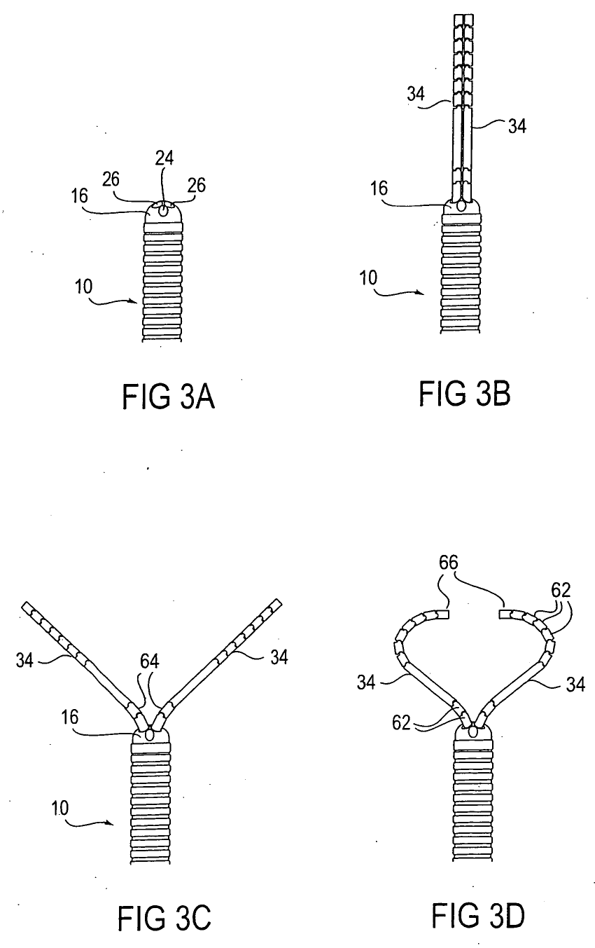

FIGS. 3A-3D illustrate a series of movements of the steerable distal ends 34 of the tool arms 30. This series serves only as an example, as a multitude of movements may be achieved by the distal ends 34 independently or together. FIG. 3A illustrates the distal tip 16 of the main body 10. The scope lumen 24 is shown along with two arm guide lumens 26 terminating at the distal tip 16 and forming a triangular pattern as illustrated in FIG. 2A. FIG. 3B illustrates the advancement of the distal ends 34 of the tool arms 30 through the arm guide lumens 26 so that the arms 30 extend beyond the distal tip 16. FIGS. 3C-3D illustrate deflection of the arms 30 to a preferred arrangement. FIG. 3C illustrates deflection of the arms 30 laterally outward. This is achieved by curvature in the outward direction near the base 64 of the steerable distal end 34. FIG. 3D illustrates deflection of the tip section 66 of the distal end 34 laterally inward achieved by curvature in the inward direction so that each arm 30 forms a hook shape. By facing the tip sections 66 of the arms 30 toward each other as shown, the tip sections 66 are positioned directly in the path of the scope lumen 24. Therefore, when a scope 28 is positioned within the scope lumen 24, the tip sections 66 of the tool arms 30 and any tools 40 advanced therethrough, will be visible through the scope 28. In FIGS. 3C-3D, deflection of the arms 30 is achieved with the use of adjacent links 62 in the areas of desired curvature. Embodiments of such links 62 and other mechanisms of deflection will be discussed in later sections. Further, the deflection of FIGS. 3A-3D are shown to be within a single plane. However, various embodiments include deflection in multiple planes. Likewise, the arms 30 are shown to be deflected simultaneously in FIGS. 3A-3D, however the arms 30 may be deflected selectively or independently.

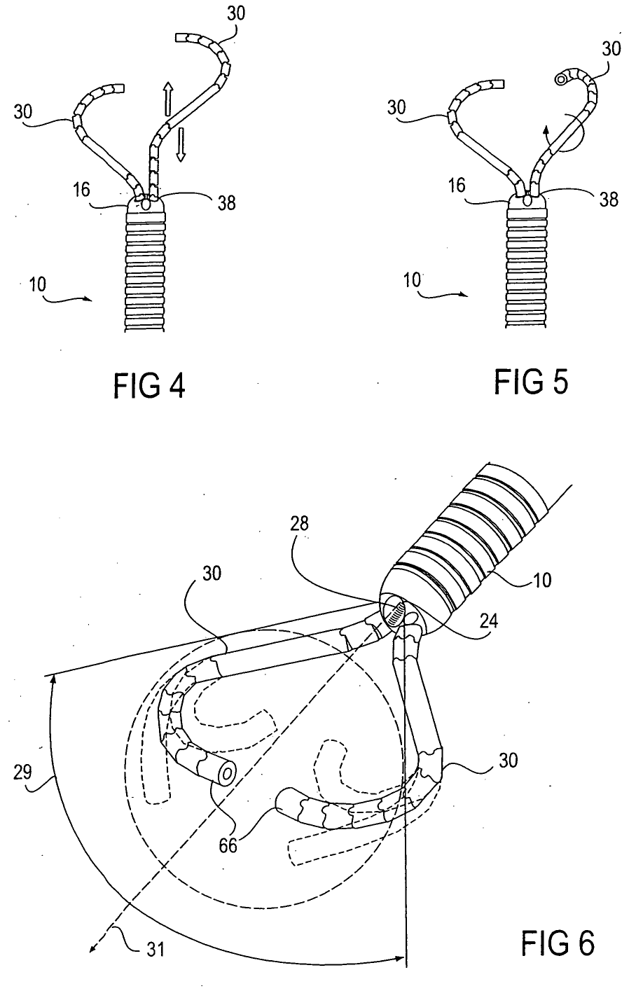

FIGS. 4-6 illustrate additional possible movements of the tool arms 30. For example, FIG. 4 illustrates axial movement of the tool arms 30. Each tool arm 30 can independently move distally or proximally, such as by sliding within the tool deployment lumen 38, as indicated by arrows. Such movement maintains the arms 30 within the same plane yet allows more diversity of movement and therefore surgical manipulations. FIG. 5 illustrates rotational movement of the tool arms 30. Each tool arm 30 can independently rotate, such as by rotation of the arm 30 within the tool deployment lumen 38, as indicated by circular arrow. Such rotation moves the arm 30 through a variety of planes. By combining axial, lateral and rotational movement, the arms 30, and therefore the tools 40 positioned therethrough (or formed integrally therewith), may be manipulated through a wide variety of positions in one or more planes.

FIG. 6 illustrates further articulation of the tool arms 30. In some embodiments, the arms 30 are deflectable to form a predetermined arrangement, such as illustrated in FIG. 3D. Typically, when forming the predetermined arrangement, the arms 30 are steerable up until the formation of the predetermined arrangement wherein the arms 30 are then restricted from further deflection. In other embodiments, the arms are deflectable to a variety of positions and are not limited by a predetermined arrangement. Such an embodiment is illustrated in FIG. 6 wherein the arms 30 articulate so that the tip sections 66 curl inwardly toward the distal tip 16 of the main body 10. Again, the tip sections 66 are positioned in front of the scope lumen 24 and scope 28 for viewing. Typically, the tip sections 66 are positioned on opposite sides of a central axis 31 of the scope 28, wherein the field of view (indicated by arrow 29) spans up to approximately 140 degrees, approximately 70 degrees on each side of the central axis 31. In addition, the depth of field is typically in the range of approximately 1-10 cm.

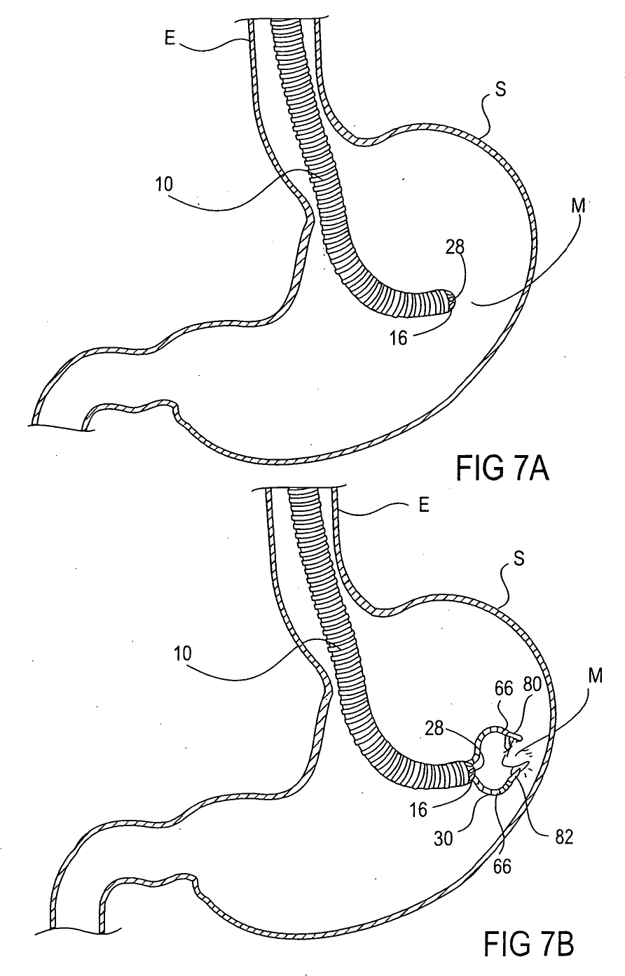

As mentioned previously, the endoluminal tool deployment system 2 of the present invention may be used to access various internal tissues or organs to perform a wide variety of surgical procedures. FIGS. 7A-7B illustrate the use of an embodiment of the system 2 to perform a mucosectomy, or removal of a portion of the mucosa and/or submucosa of the stomach. FIG. 7A illustrates advancement of the main body 10 through the esophagus E to the stomach S. The main body 10 is then steered to a desired position within the stomach S and the stomach mucosa M is visualized through the scope 28 at the distal tip 16. Referring to FIG. 7B, the tool arms 30 are then advanced through the main body 10 and articulated. As mentioned, tools 40 may be advanced through the tool arms 30 or an end effector 48 may be disposed at the distal end of each arm 30. Here, a grasper 80 is disposed at the distal end of one arm 30 and a cutter 81 is disposed at the distal end of the other arm 30. The grasper 80 is used to grasp a portion of the mucosa M. The grasped portion of mucosa M can then be elevated by rotation or manipulation of the tool arm 30. This allows safe resection of the portion of mucosa M by cutting with the use of the cutter 82, as shown. Manipulation and resection of the tissue is visualized throughout the procedure through the scope 28, which is aligned with the tip sections 66 and therefore with end effectors 48.

It may be appreciated that the systems, methods and devices of the present invention are applicable to diagnostic and surgical procedures in any location within a body, particularly any natural or artificially created body cavity. Such locations may be disposed within the gastrointestinal tract, urology tract, peritoneal cavity, cardiovascular system, respiratory system, trachea, sinus cavity, female reproductive system and spinal canal, to name a few. Access to these locations may be achieved through any body lumen or through solid tissue. For example, the stomach may be accessed through an esophageal or a port access approach, the heart through a port access approach, the rectum through a rectal approach, the uterus through a vaginal approach, the spinal column through a port access approach and the abdomen through a port access approach.

A variety of procedures may be performed with the systems and devices of the present invention. The following procedures are intended to provide suggestions for use and are by no means considered to limit such usage: Laryngoscopy, Rhinoscopy, Pharyngoscopy, Bronchoscopy, Sigmoidoscopy (examination of the sigmoid colon, the sigmoid colon is the portion that connects the descending colon to the rectum; primarily for diagnostic purposes, however a biopsy procedure and trans anal micro surgery may be performed for removing tumors), Colonoscopy (examination of colon; for the removal of polyps and tumors or for biopsy), and Esophagogastroduodenoscopy (EGD) which enables the physician to look inside the esophagus, stomach, and duodenum (first part of the small intestine). The procedure might be used to discover the reason for swallowing difficulties, nausea, vomiting, reflux, bleeding, indigestion, abdominal pain, or chest pain.

In addition, endoscopic retrograde cholangiopancreatography (ERCP) may be achieved which enables the surgeon to diagnose disease in the liver, gallbladder, bile ducts, and pancreas. In combination with this process endoscopic sphincterotomy can be done for facilitating ductal stone removal. ERCP may be important for identification of abnormalities in the pancreatic and biliary ductal system. Other treatments include Cholecystectomy (removal of diseased gallbladder), CBD exploration (for common bile duct stones), appendicectomy (removal of diseased appendix), hernia repair TAP, TEPP and other (all kinds of hernia), findoplication and HISS procedures (for gastro esophageal reflux disease), repair of duodenal perforation, gastrostomy for palliative management of late stage upper G.I.T. carcinoma), selective vagotomy (for peptic ulcer disease), splenectomy (removal of diseased spleen), upper and lower G.I. endoscopies (diagnostic as well as therapeutic endoscopies), pyloroplastic procedures (for children's congenital deformities), colostomy, colectomy, adrenalectomy (removal of adrenal gland for pheochromocytoma), liver biopsy, gastrojejunostomy, subtotal liver resection, gastrectomy, small intestine partial resections (for infarction or stenosis or obstruction), adhesions removal, treatment of rectum prolaps, Heller's Myotomy, devascularization in portal hypertension, attaching a device to a tissue wall and local drug delivery to name a few.

It is expected that the systems and devices of the present invention will have significant utility in gastric restrictive and/or malabsorbtive procedures for morbid obesity, such as endoluminal banded gastroplasty. Furthermore, the systems and devices are particularly suited to tissue plication procedures. Illustrative plication and endoluminal gastric restriction/reduction methods and apparatus are described, for example, in Applicant's copending U.S. patent application Ser. No. 10/735,030, filed Dec. 12, 2003, from which the present application claims priority and which is incorporated herein by reference.

II. Main Body

As mentioned previously, the system 2 of the present invention includes an elongated main body 10 having a proximal end 12 and a distal end 14 terminating in a distal tip 16. For ease of description, the main body 10 will be described in terms of sections, including one or more of section A, section B and section C. Section A refers to a deflectable and/or steerable portion which may be advanced through supported or unsupported anatomy. Section B refers to a portion which is capable of retroflexion. Typically, this section is laterally stabilized and deflectable in a single plane. Thus, section B is ideal for steering within open cavities. Section C refers to a steerable portion, typically steerable within any axial plane in a 360 degree circumference around the shaft. When section C is the most distal section, such steerability allows movement of the distal tip in a variety of directions. Such sections will be further described below. It may be appreciated that the main body 10 may be comprised of any combination of sections A, B, and C and may include such sections in any arrangement. Likewise, the main body 10 may be comprised of any subset of sections A, B, an C, such as section A and section C, or simply section C. Further, additional sections may be present other than sections A, B, and C. Furtherstill, multiple sections of a given variety, e.g. multiple B sections, may be present. And finally, sections A, B, C may be independently lockable, as will be described below.

A-B Sectioned Embodiment

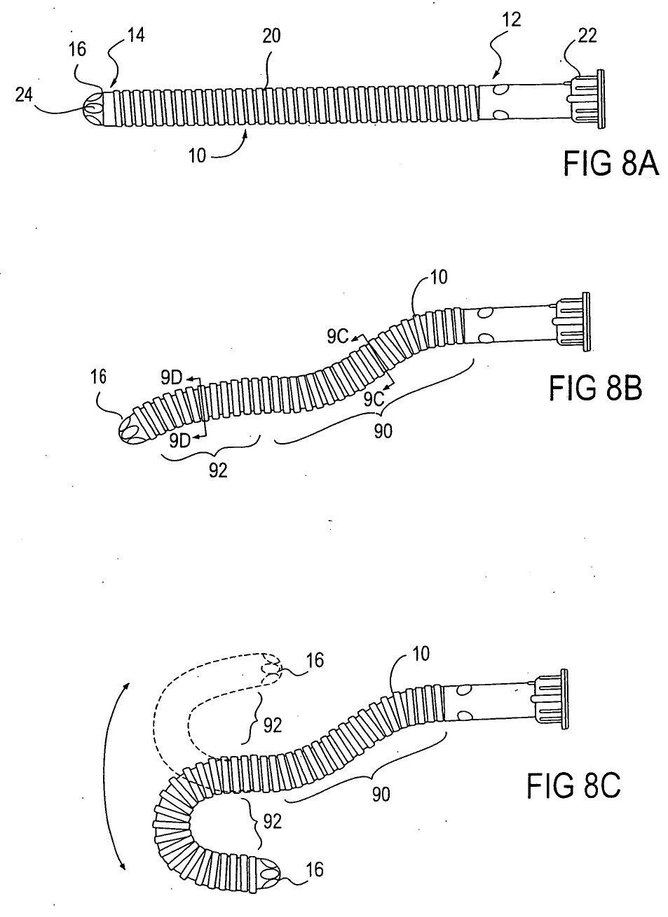

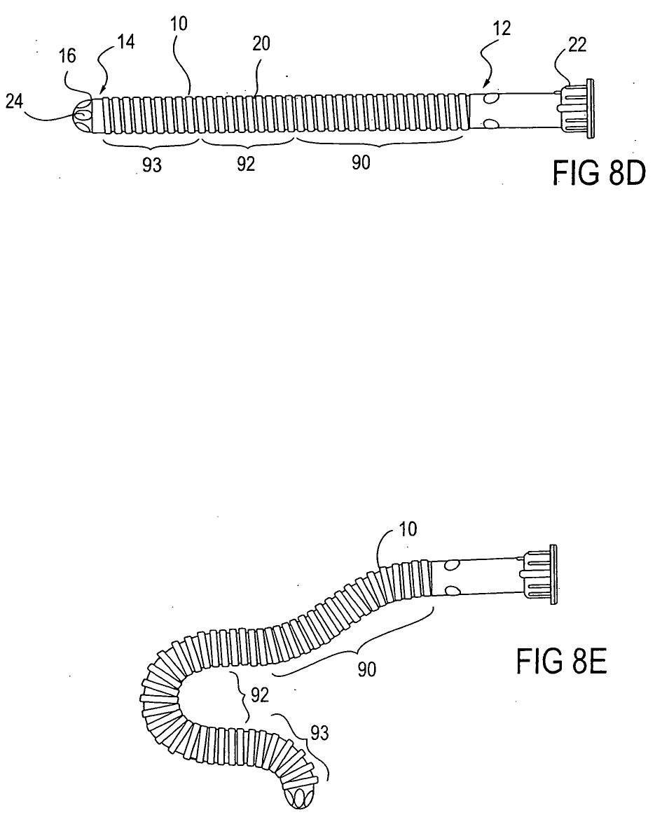

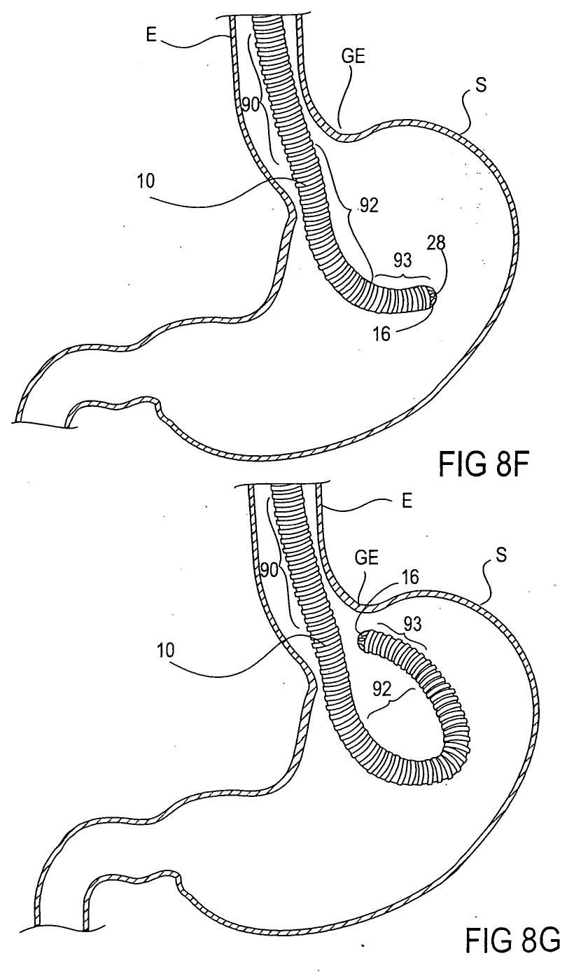

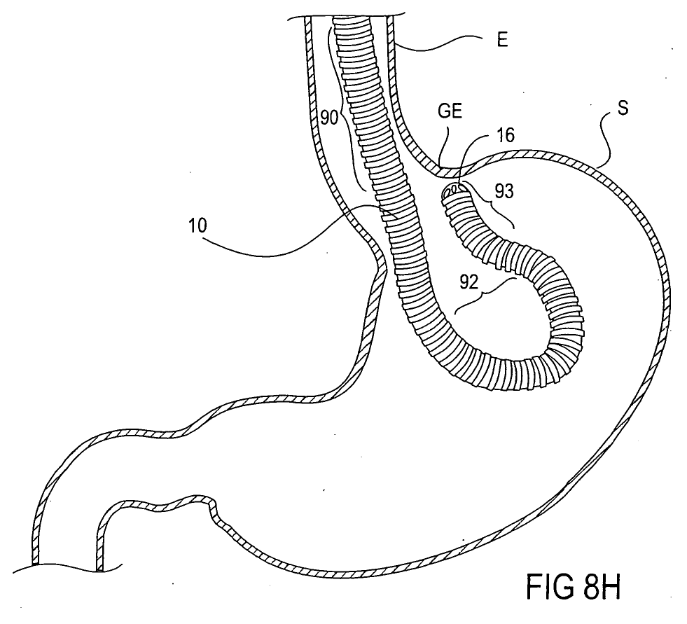

An embodiment of the main body 10 is illustrated in FIG. 8A in a straight configuration. However, the main body 10 is used to access an internal target location within a patient's body so the main body 10 has a deflectable and/or steerable shaft 20. Thus, FIG. 8B illustrates the main body 10 having various curvatures in its deflected or steered state. In preferred embodiments, the main body 10 is steerable so that the main body 10 may be advanced through unsupported anatomy and directed to desired locations within hollow body cavities. In this embodiment, the main body 10 includes a first section 90 (section A) which is proximal to a second section 92 (section B), as indicated in FIG. 8B. Although both sections 90, 92 are steerable, the first section 90 may be locked in place while the second section 92 is further articulated. This is illustrated in FIG. 8C, wherein the first section 90 is shown in a locked position unchanged from FIG. 8B and the second section 92 is shown in various retroflexed positions. In retroflexion, the second section 92 is curved or curled laterally outwardly so that the distal tip 16 is directed toward the proximal end 12 of the main body 10. In retroflexion, the section 92 may form an arc which traverses approximately 270 degrees. Likewise, the arc may have a radius of curvature between about 5 and 12 centimeters. Optionally, the second section 92 also may be locked, either in retroflexion or in any other position. It should be understood, section 90 optionally may not be steerable or lockable. For example, section 90 may comprise a tube extrusion of appropriate durometer.

A-B-C Sectioned Embodiment

Another embodiment of the main body 10 is illustrated in FIG. 8D, in a straight configuration, and in FIG. 8E, in a deflected or steered state having various curvatures. In this embodiment, the main body 10 includes a first section 90 (section A) which is proximal to a second section 92 (section B), which is proximal to a third section 93 (section C). The first section 90 may be flexible or semi-flexible, e.g. such that the section 90 is primarily moveable through supported anatomy or is moveable through unsupported anatomy via one or more stiffening members disposed within or about the section. The first section 90 may be comprised of a solid plastic or polymeric material, such as polyurethane, nylon, or polyvinyl chloride (PVC). Alternatively, the first section 90 may be comprised of links or nestable elements. The first section 90 is typically deflectable, however it may optionally be steerable, such as with the use of pullwires.

Optionally, the first section 90 may include locking features for locking the section in place while the second section 92 is further articulated. Typically, the second section 92 is capable of retroflexion. In retroflexion, as illustrated in FIG. 8E, the second section 92 is curved or curled laterally outwardly so that a portion of the second section 92 is directed toward the proximal end 12 of the main body 10 or toward shaft 20 of the main body. It may be appreciated that the second section 92 may be retroflexed in any desired direction. Optionally, the second section 92 may also be locked, either in retroflexion or in any other position.

Further, the first section 90 and second section 92 preferably may be locked in place while the third section 93 is further articulated. Such articulation is typically achieved by steering, such as with the use of pullwires. The distal tip 16 preferably may be steered in any direction relative to the second section 92. For example, with the second section 92 defining an axis, the third section 93 may move within an axial plane, such as in a wagging motion. The third section 93 preferably may move through any axial plane in a 360 degree circumference around the axis, thus the third section 93 may wag in any direction. Further, the third section 93 may be further steerable to direct the distal end within any plane perpendicular to any of the axial planes. Thus, rather than wagging, the distal end may be moved in a radial fashion, such as to form a circle around the axis. FIG. 8E illustrates the third section 93 steered into an articulated position within an axial plane.

The embodiment of FIGS. 8D-8E having three sections 90, 92, 93 with varying movement capabilities as described is particularly useful for accessing desired target regions within a patient's stomach. FIGS. 8F-8H illustrate positioning of this embodiment of the main body 10 within a stomach S through an esophagus E. Since the main body 10 is deflectable and at least some of the sections 90, 92, 93 preferably are steerable, the main body 10 may be advanced through the tortuous or unpredictably supported anatomy of the esophagus and into the stomach while reducing a risk of distending or injuring the organs, as shown in FIG. 8F. Once the distal tip 16 has entered the stomach, the second section 92 may be retroflexed as illustrated in FIG. 8G. During retroflexion, the distal tip 16 traverses an arc of substantially continuous radius of curvature. In a preferred embodiment, the arc traverses approximately 270 degrees and has a radius of curvature between about 5 and 10 cm, and, even more preferably, approximately 7-8 cm. By retroflexing about 270 degrees, distal tip 16 is directed back towards the first section 90 near and inferior to gastroesophageal junction GE. The second section 92 may be actively retroflexed, e.g. via pullwires, or may be passively retroflexed by deflecting the section off a wall of stomach S while advancing main body 10.

The second section 92 is preferably shape-lockable in the retroflexed configuration. The distal tip 16 may then be further articulated and directed to a specific target location within the stomach. For example, as shown in FIG. 8H, the distal tip 16 may be steered toward a particular portion of the gastroesophageal junction GE. Then, the third section 93 may optionally be shape-locked in this configuration. Various instruments, such as surgical tools and/or visualization scopes, may then be used to perform a procedure at the target location. The instruments may be passed through the main body 10 or may be built in to the main body 10. Shape-locking reduces the magnitude of forces required to advance and retract instruments through main body 10 and/or creates a rigid platform for use in performing the procedure at the target location. Typical procedures for such positioning would include plication near the gastroesophageal junction GE, for example, to achieve endoluminal gastric restriction or reduction.

Additional examples of medical procedures and instruments which may be used with the main body 10, as described above or in other embodiments, are provided in copending U.S. patent application Ser. No. 10/735,030 filed Dec. 12, 2003, which is incorporated herein by reference for all purposes.

III. Main Body Section Construction

The sections 90, 92, 93 (sections A, B, C) of the main body 10 may have any suitable construction, and steering and locking may be achieved by any suitable mechanisms. For example, any of the links and/or steering, locking or torqueing mechanisms provided in U.S. patent application Ser. No. 10/346,709 may be used. In addition, a few example embodiments are provided herein. It may be appreciated that the following embodiments may be used to construct any or all of the sections 90, 92, 93, however some embodiments may be more suited for particular sections, as will be described. It may also be appreciated that two or more sections 90, 92, 93 may have the same construction.

Nested Links

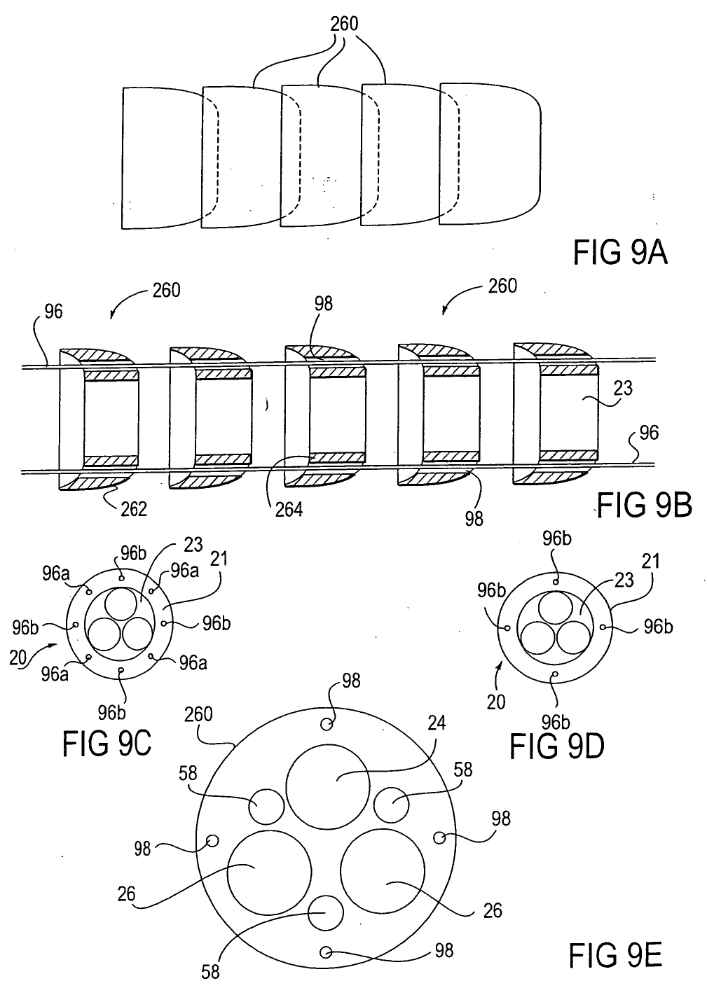

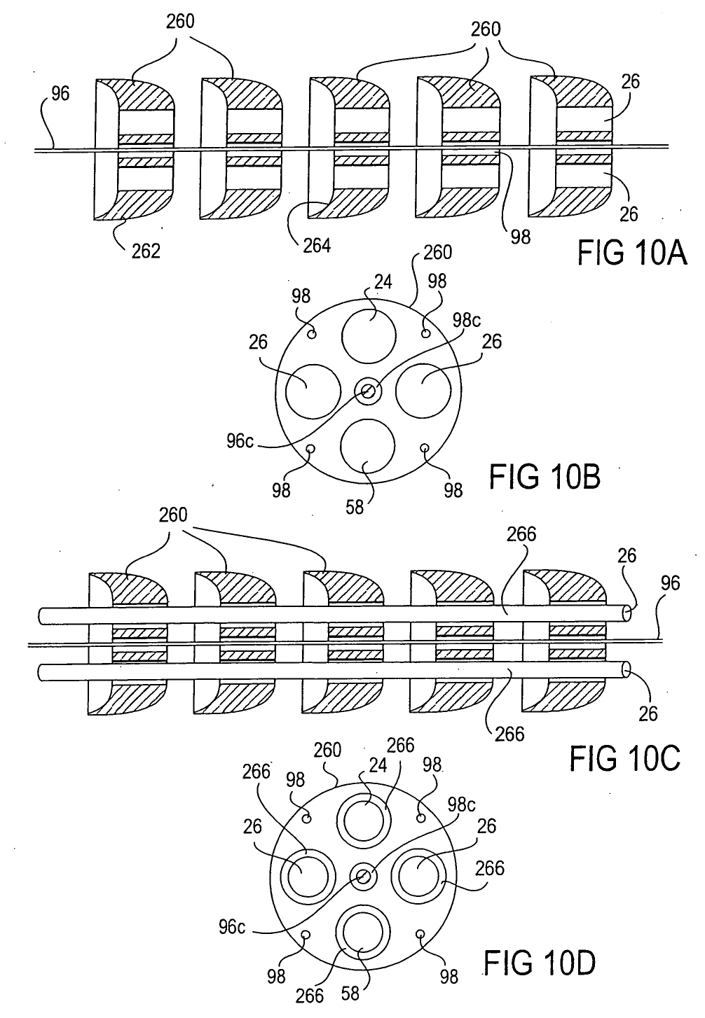

In some embodiments, one or more of the sections 90, 92, 93 of the shaft 20 of the main body 10 comprise a multiplicity of nested links or nestable elements 260, as illustrated in FIG. 9A. FIG. 9B provides an exploded view of the nestable elements 260 of FIG. 9A. Here it can be seen that the elements 260 are disposed so that a distal surface 262 of one element 260 coacts with a proximal surface 264 of an adjacent element. Each of the nestable elements 260 includes one or more pullwire lumens 98 through which pullwires 96 pass. The pullwires 96 are used to hold the elements 260 in nesting alignment and to provide steering and locking. The pullwires 96 preferably are made from a superelastic material, e.g. nickel titanium alloy, to provide flexibility, kink-resistance and smooth movement of the pullwires 96 through the pullwire lumens 98. Alternatively, the pullwires 96 may be made from braided stainless steel, a single stainless steel wire, poly-para-phenylene terephthalamide (such as Kevlar®), a high tensile strength monofilament thread, combinations thereof or any suitable materials.

Generally, the adjacent surfaces 262, 264 are contoured to mate so that when the pullwires 96 are relaxed, surfaces 262, 264 can rotate relative to one another. This allows the shaft 20 to form curvatures throughout its length in any direction. Each pullwire 96 is fixed at its distal end to a specific element 260 along the shaft 20 or to the distal tip 16. When tension is applied to a specific pullwire 96, a curvature forms in the shaft 20 proximal to the fixation point, thus steering the shaft 20. The pullwires 96 may be arranged in various patterns to achieve steering in various directions.

For example, FIG. 9C is a cross-sectional view of the shaft 20 in the first section 90 of FIG. 8B. Here, eight pullwires 96 (four pullwires 96a and four pullwires 96b) are shown passing through the wall 21. Four pullwires 96a terminate at the distal end of the first section 90 and are used to steer the first section 90. Since the pullwires 96a are equidistantly positioned, applying tension to the pullwires 96a, either individually or in combination, steers the first section 90 in any desired direction. The first section 90 may be locked in place by holding the tension in the pullwires 96a using any suitable mechanisms. For example, tension may be applied to the pullwires 96 simultaneously until the elements 260 are compressed to a state in which they are locked by friction wherein the tension is held.

FIG. 9D is a cross-sectional view of the shaft 20 in the second section 92 of FIG. 8B. Here, four pullwires 96b are shown passing through the wall 21. These pullwires 96b extended through the first section 90, as indicated in FIG. 9C, and terminate near the distal tip 16. Since the pullwires 96b are equidistantly positioned, applying tension to the pullwires 96b, either individually or in combination, steers the second section 92 in any desired direction. Since the pullwires 96b also pass through the first section 90, such steering may also effect the curvature in the first section 90 when the first section is not locked. However, such effects may be reduced, counteracted or compensated for by steering in the first section 90 or by locking. The second section 92 may be also be locked in place by holding the tension in the pullwires 96b using any suitable mechanisms.

In this embodiment, the wall 21 extends continuously from the proximal end 12 to the distal end 14 with the first and second sections 90, 92 determined by the termination points of the pullwires 96 which extend therethrough. It may be appreciated that although the first and second sections 90, 92 have been used in this example, the above description is also applicable to the second and third sections 92, 93. Or, the nestable elements 260 may be used to form a single section.

In the embodiment illustrated in FIG. 9B, the nestable elements 260 have a central lumen 23 which passes through the length of the main body 10. Instruments or tools may be passed through this lumen 23, as indicated in FIGS. 9C-9D, or tubes may be present within the lumen 23 through which instruments or tools may be passed. In preferred embodiments, the nestable elements 260 have holes formed therein so that lumens are formed by alignment of the holes when the elements 260 are stacked. For example, FIG. 9E provides a cross-sectional view of a nestable element 260 illustrating the holes formed therein which serve as lumens. As shown, a scope lumen 24, arm guide lumens 26, and auxiliary lumens 58 extend through the center of the element 260 while pullwire lumens 98 are located around the periphery. It may be appreciated that pullwire lumens 98 may also extend through the center of the element 260. For example, FIG. 10A illustrates an embodiment having a pullwire 96 which extends through the center of the stacked nestable elements 260. FIG. 10A provides an exploded view of the nestable elements 260 wherein the elements 260 are disposed so that a distal surface 262 of one element 260 coacts with a proximal surface 264 of an adjacent element. As shown, each of the nestable elements 260 includes a pullwire lumen 98 through its center. FIG. 10B provides a cross-sectional view of a nestable element 260 of FIG. 10A. As shown, the nestable element 260 includes a locking pullwire lumen 98c having a pullwire 96c therethrough in the center of the element 260 surrounded by various other lumens, such as a scope lumen 24, arm guide lumens 26, auxiliary lumen 58 and various pullwire lumens 98 used for steering. Once the elements 260 are positioned in a desired arrangement, the shaft 20 may be locked in place by the central pullwire 96c. Applying tension to the pullwire 96c compresses the elements 260 to a state in which they are locked by friction wherein the tension is held.

In addition, liners 266 may be passed through any of the lumens of the stacked nestable elements 260. Such liners 266 form create a continuous lumen connecting the lumen holes of the nestable elements 260. FIG. 10C illustrates the nestable elements 260 of FIG. 10A with the inclusion of liners 266 passing through, for example, the arm guide lumens 26. Likewise, FIG. 10D provides a cross-sectional view of a nestable element 260 of FIG. 10C. Here, liners 266 are shown positioned through the nestable element 260 forming lumens 24, 26, 58 therethrough. It may also be appreciated that liners 266 may extend through pullwire lumens 98 as well: The liners 266 may be coated on their luminal surface with a hydrophilic coating for reducing friction or the liners 266 may be comprised of a lubricious polymer such as Teflon®, fluoroethylene polymer (FEP) or the like. Additionally or alternatively, the liners may be torqueable, e.g. fabricated from a coil.

Bump Links

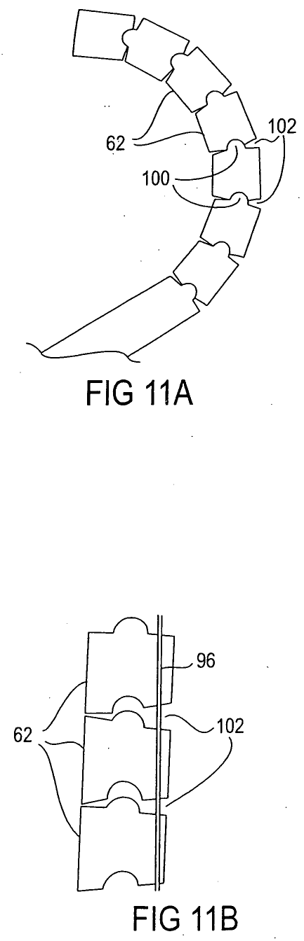

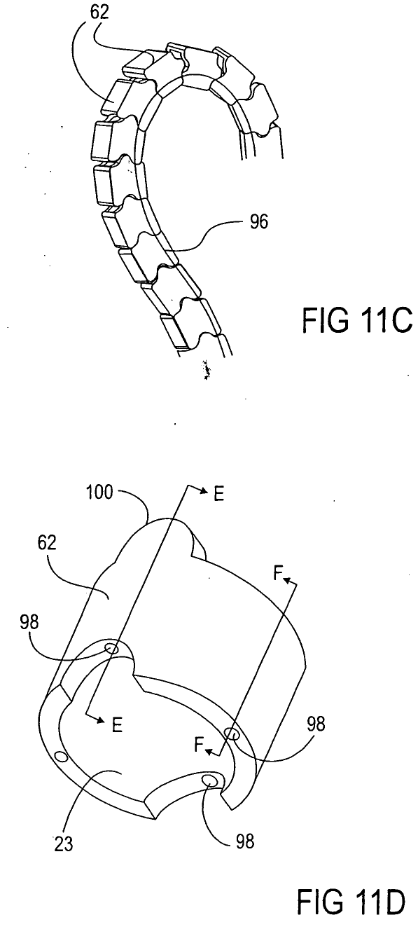

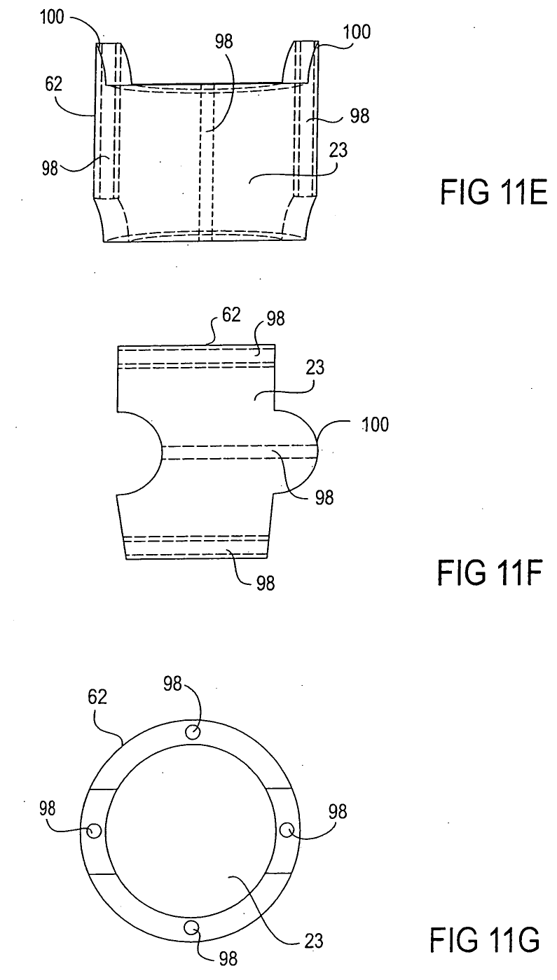

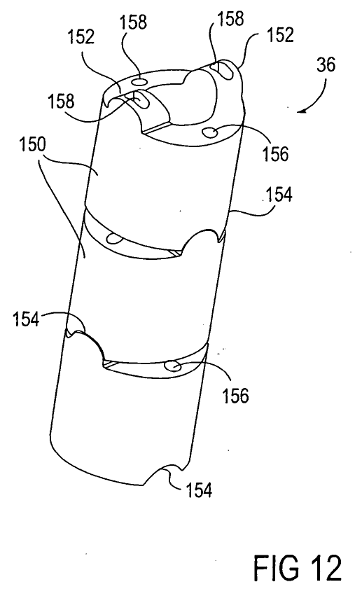

In some embodiments, one or more of the sections 90, 92, 93 of the shaft 20 of the main body 10 comprise a multiplicity of bump links 62, as illustrated in FIGS. 11A-11G and FIG. 12. Bump links 62 may be used to allow steering of a section 90, 92, 93 to a predetermined arrangement, such as a continuous radius of curvature during retroflexion, or to allow steering in a variety of directions.

FIGS. 11A-11B illustrates an embodiment wherein the bump links 62 are steerable to a predetermined arrangement. FIG. 11A is an enlarged view which illustrates the shapes of the links 62 which are pivotally connected by hinge structures 100 formed into the links 62. Gaps 102 are present on opposite sides of the structures 100 to allow curvature. The size of the gaps 102 will vary due to varying sizes and shapes of the links 62 so that closure of the gaps 102 forms a specific curvature. This is most easily seen in FIGS. 11B-11C. FIG. 11B illustrates links 62 having varying shapes to create gaps 102 of varying size. As shown, a pullwire 96 extends through the links 62 along the gaps 102. Applying tension to the pullwire 96 draws the links 62 together to close the gaps 102 and to form a predetermined curve as in FIG. 11C.

FIGS. 11D-11G illustrate an exemplary bump link 62. FIG. 11D provides a perspective view. A hinge structure 100 is shown on one end and it may be appreciated that another hinge structure is present in a location symmetrically opposite to the visible hinge structure 100. In addition, pullwire lumens 98 are shown through which pullwires 98 may be positioned. Further, FIG. 11D illustrates an open central lumen 23. FIG. 11E provides a cross-sectional view along line E-E of FIG. 11D. And, FIG. 11F provides a cross-sectional view along line F-F of FIG. 11D. FIG. 11G provides a bottom view.

FIG. 12 illustrates an embodiment wherein the bump links are steerable in a variety of directions. Here, each linkage 150 includes a pair of protruding structures 152 on its face and a pair of notches 154 on its base. The protruding structures 152 and notches 154 are both arc shaped so that the protruding structures 152 of one linkage 150 rotateably interfit with the notches 154 of an adjacently stacked linkage 150. By alternating the position of the pairs of protruding structures 152 and notches 154 as shown in FIG. 12, the shaft 36 is flexible in both lateral bending directions while maintaining stiffness axially and in torsion. Also shown are flared lumens 158 which pass through the protruding structures 152 and the wall of the shaft 36. Flaring allows for a rod or wire passed therethrough to move within the lumen 158 as a linkage 150 rotates over the protruding structure 152. Round pullwire lumens 156 pass through the notches 154 and the wall of the shaft 36 as shown. The rod or wire holds the linkages 150 in a stacked configuration and optionally may be used to steer the shaft 36.

Pin Link

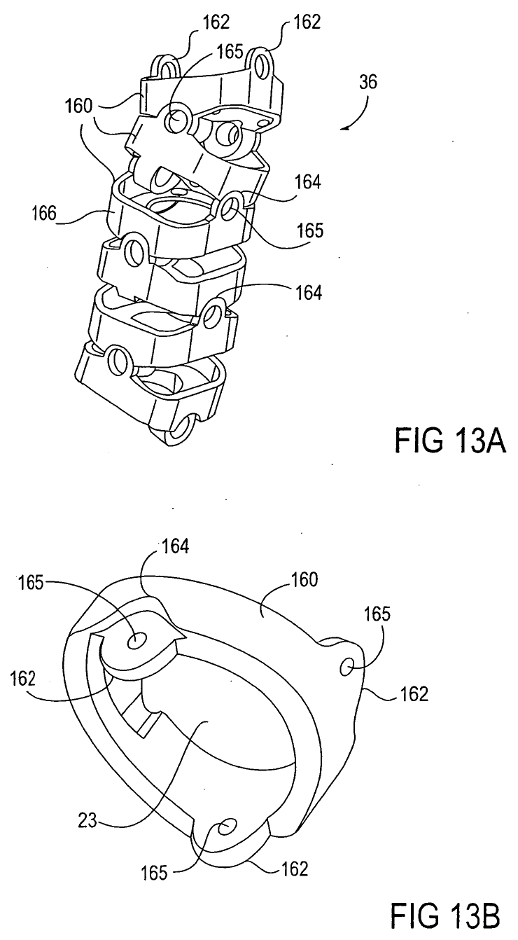

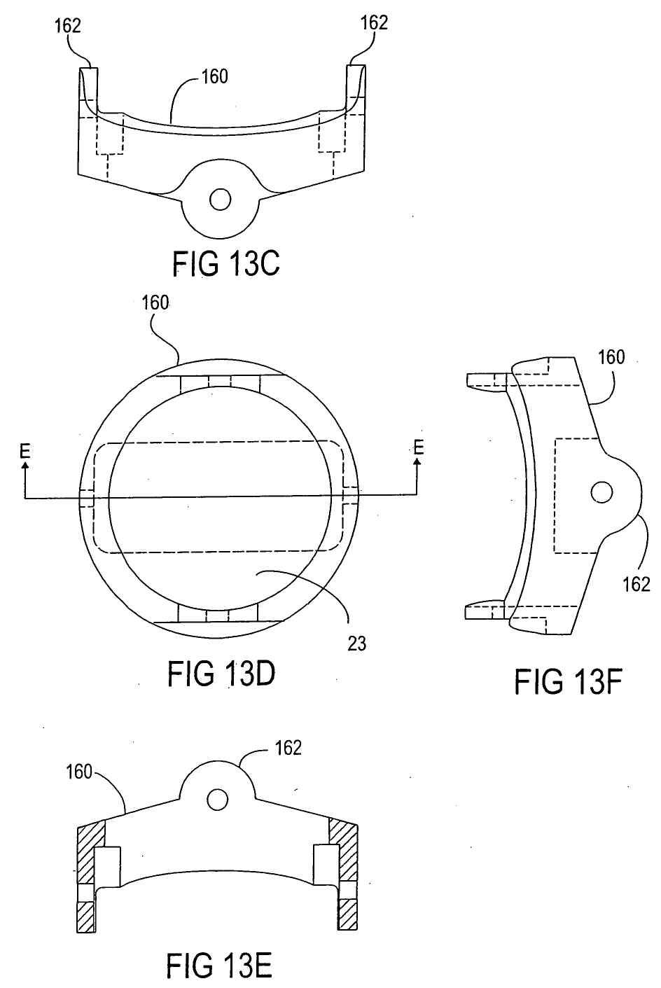

In some embodiments, one or more of the sections 90, 92, 93 of the shaft 20 of the main body 10 comprise a multiplicity of pin links 160, as illustrated in FIG. 13A. Here, the shaft 36 comprises a plurality of adjacent linkages 160 which are stacked to provide lateral deflection while maintaining axial rigidity. Here, each linkage 160 includes a pair of protruding structures 162 on its face and a pair protruding structures 162 with notches 164 on its base. The notches 164 are arc shaped so that the protruding structures 162 of one linkage 160 rotateably interfit with the notches 164 of an adjacently stacked linkage 160. Pins fit through holes 165 in the protruding structures 162 to hold the linkages 160 together. By alternating the position of the pairs of protruding structures 162 and notches 164 as shown in FIG. 13A, the shaft 36 is flexible in both lateral bending directions while maintaining stiffness axially and in torsion. In this embodiment, the linkages 150 include a central lumen 166 through which a rod or wire is passed. The rod or wire is used to hold the linkages 60 in the stacked configuration.

FIGS. 13B-13F illustrate an exemplary pin link 160. FIG. 13B provides a perspective view. Three protruding structures 162 are shown and it may be appreciated that another protruding structure is present in a location symmetrically opposite the protruding structure 162 on the face. Further, FIG. 13B illustrates an open central lumen 23. FIG. 13C provides a side view of the link 160 between the protruding structures 162. FIG. 13D provides a bottom view. FIG. 13E provides a cross-sectional view along line E-E of FIG. 13D. And, FIG. 13F provides a side view of the link 160 facing a protruding structure 162.

Pinned Nested Links

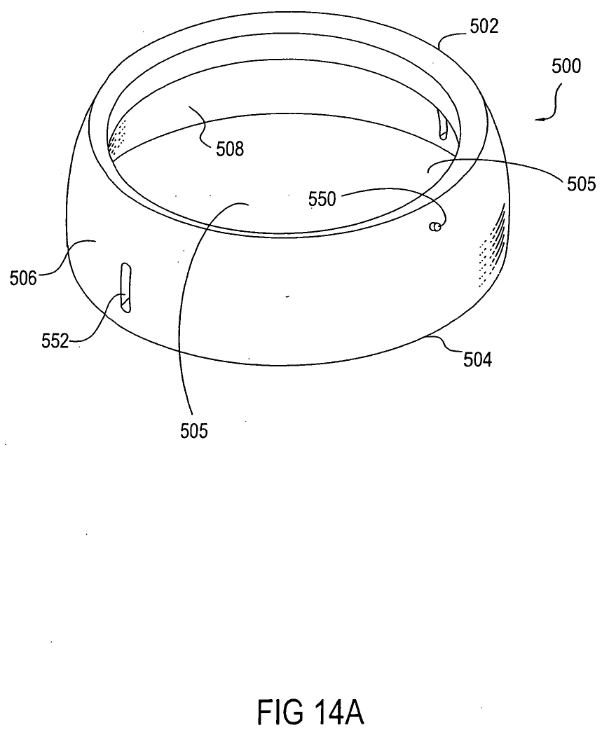

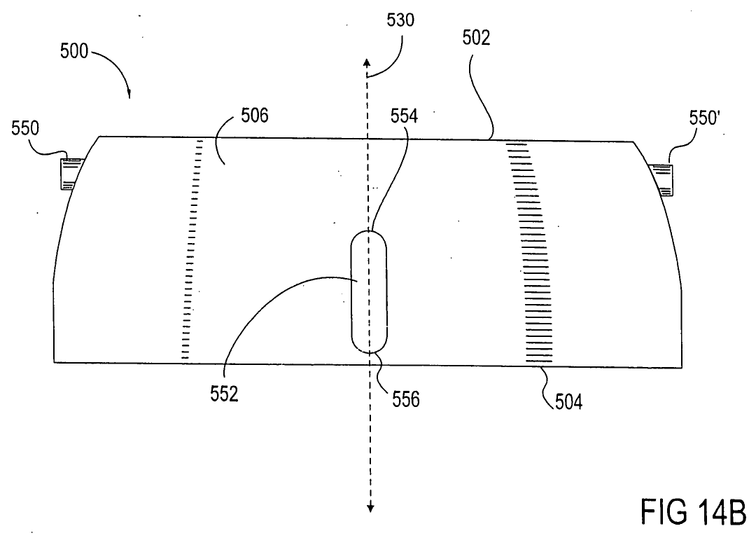

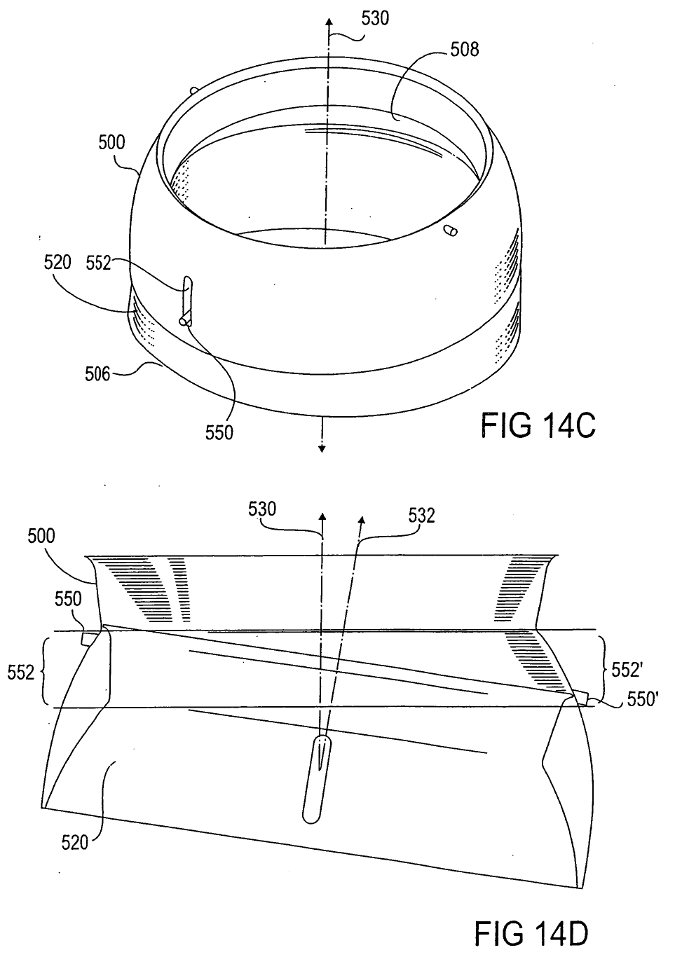

In some embodiments, one or more of the sections 90, 92, 93 of the shaft 20 of the main body 10 comprise a multiplicity of pinned nested links. Pinned nested links have a torque transmitting feature as illustrated in FIGS. 14A-14D. FIGS. 14A-14D illustrate the use of a pin and slot concept to maintain alignment of the plurality of adjacent links at locations along its length. By maintaining alignment in particular locations, torque may be more easily transmitted while still allowing freedom of rotation of the links for steering. In addition, the pin and slot concept prevents disengagement of the adjacent links while the main body is unlocked. This further enhances torque transmission.

FIG. 14A is a perspective view of one of the plurality of adjacent links, a first link 500. The first link 500 has a top edge 502, a bottom edge 504, an outer surface 506 and an inner surface 508 forming a domed ring-like structure having a lumen 505 therethrough. Although pullwire lumens are not shown, it may be appreciated that pullwire lumens may be present, for example passing through the inner surface and out through the top edge. It may also be appreciated that the pullwire lumens may be used for other elements, such as support wires or rigidizing wires, however at least some of the pullwire lumens are used for passing pullwires for steering. The first link 500 also includes a torque transmitting feature comprising at least one protrusion, such as a pin 550, which protrudes outward from the outer surface 506. The torque transmitting feature also includes at least one slot 552, providing an opening between the inner surface 508 and the outer surface 506.

In some embodiments, a pair of pins 550, 550′ are present wherein one pin 550 is located in a diametrically opposite position from the other pin 550′. Likewise, a pair of slots 552, 552′ are also present wherein one slot 552 is located in a diametrically opposite position from the other slot 552′. Typically, the pair of pins 550, 550′ and pair of slots 552, 552′ are located approximately 90 degrees apart.

FIG. 14B provides a side view of the first link 500 of FIG. 14A. It may be appreciated that the pin 550 may have any suitable shape or size and may be positioned anywhere along the outer surface 506. In this embodiment, the pins 550, 550′ each have a cylindrical shape with a cross-sectional diameter of approximately 0.0325 in and each is positioned near the top edge 502. Each slot 552 is sized, shaped and positioned so that when the first link 500 is engaged with an adjacent link, a slot 552 in the first link 500 accepts a pin 550 on the adjacent link. Typically, each slot 552 is positioned near the bottom edge 504, preferably 0.010 in from the bottom edge 504 as illustrated in FIG. 14B. Also illustrated in FIG. 14B, each slot 552 has a first slot end 554 and a second slot end 556, typically approximately 0.090 in. apart. The slot ends 554, 556 are substantially aligned with the longitudinal axis 530 to allow sliding of the pin 550 through the slot during rotation of the link away from the longitudinal axis 530, as will be illustrated in FIGS. 14C-14D.

FIG. 14C illustrates the first link 500 engaged with a second link 520 having the same or similar features as the first link 500. The links 500, 520 are each individually rotateable by steering, such as with the use of pullwires 96 (not shown) as described in related earlier sections. As shown, the outer surface 506 of each link is mated with the inner surface 508 of an adjacent link along a longitudinal axis 530. The first link 500 is shown to have a pair of slots 552, 552′, of which one slot 552 is visible in this view. Extending through the one slot 552 is a pin 550 which protrudes from the outer surface 506 of the adjacent second link 520. It may be appreciated that the second link 520 also has an additional pin 550′ which passes through slot 552′.

Steering rotates at least some of the links away from the longitudinal axis 530, such as illustrated in FIG. 14D. Here, the first link 500 is shown rotated along another axis 532 which forms an angle with the longitudinal axis 530. Such rotation slides one pin 550 on the second link 520 upward along the slot 552 in the first link 500 while another pin 510′ slides downward along the slot 552′ in the first link 500. Thus, the second link 520 is free to rotate in this plane. It may be appreciated that each link is free to rotate in at least a plane defined by the alignment of pins and slots. When the position of such aligned pins and slots are varied along the length of the plurality of adjacent links, the links are able to rotate in various directions.

In addition, torqueing of the plurality of adjacent links is transmitted through the aligned pins and slots. For example, by applying torque to the second link 520, the second link 520 will rotate about the longitudinal axis 530 until one of the slots contacts the slidably engaged pin that transmits the torque to the first link 500. This transmission may be repeated through any number of links, transmitting torque through a plurality of adjacent links.

Saddle Links

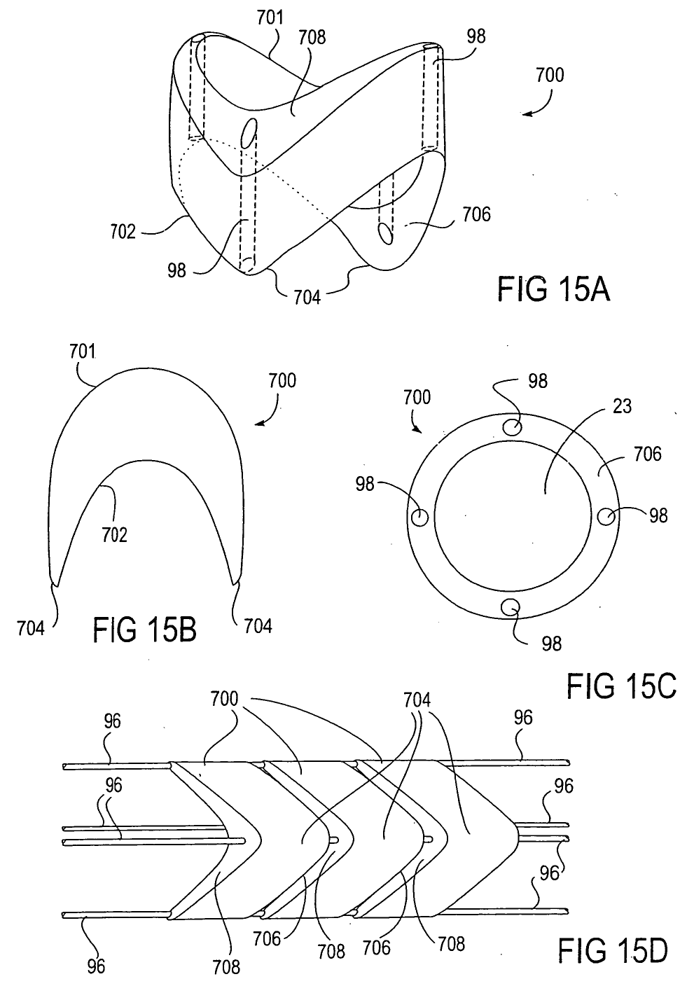

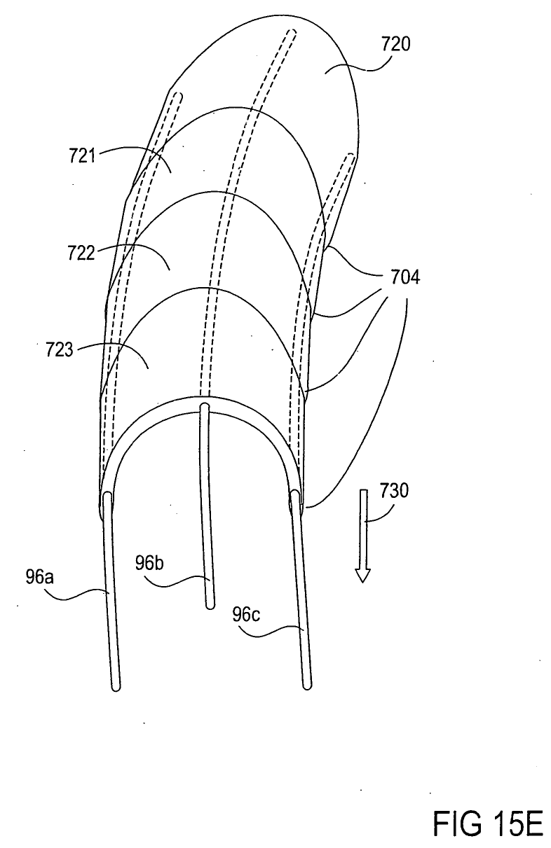

In some embodiments, one or more of the sections 90, 92, 93 of the shaft 20 of the main body 10 comprise a multiplicity of saddle links. FIG. 15A provides a perspective view of an embodiment of a saddle link 700 having a top edge 701 and a bottom edge 702. Here, the saddle link 700 includes two flanges 704, each flange 704 extending in the same direction on opposite sides of the saddle link 700 forming a curvature of the bottom edge 702. Beveling of the inside of the link 700 along the bottom edge 702 forms a bottom mating surface 706. The top edge 701 follows a curvature which is similar yet less dramatic than the bottom edge 702. Beveling of the outside of the link 700 along the top edge 701 forms a top mating surface 708. In addition, pullwire lumens 98 are shown extending from the top mating surface 708 to the bottom mating surface 706 through each flange 704. In addition, pullwire lumens 98 are present extending through the wall of the link 700 at locations equidistant between the flanges 704. FIG. 15B provides a side view of the saddle link 700 of FIG. 15A, and FIG. 15C provides a bottom view of the saddle link 700 of FIG. 15A.

Stacking of a plurality of saddle links 700 is shown in FIG. 15D. Here it can be seen that the links 700 are disposed so that a bottom mating surface 706 of link 700 coacts with a top mating surface 708 of an adjacent link. And, as shown, each of the links 700 includes one or more pullwire lumens 98 through which pullwires 96 pass. The pullwires 96 are used to hold the links 700 in nesting alignment and to provide steering and locking. The pullwires 98 preferably are made from a superelastic material, e.g. nickel titanium alloy, to provide flexibility, kink-resistance and smooth movement of the pullwires 96 through the pullwire lumens 98. Alternatively, the pullwires 96 may be made from braided stainless steel, a single stainless steel wire, poly-para-phenylene terephthalamide (such as Kevlar®), a high tensile strength monofilament thread, combinations thereof or any suitable materials.

Generally, the adjacent surfaces 706, 708 are contoured to mate so that when the pullwires 96 are relaxed, surfaces 706, 708 can rotate relative to one another. The flanges 704 allow rotation of the links 700 in the direction of the flanges 704 (i.e. along the exposed top mating surfaces 708 in FIG. 15D), yet limit rotation of the links 700 in other directions. Such rotation can be seen in FIG. 15E. FIG. 15E illustrates a first saddle link 720, a second saddle link 721, a third saddle link 722, and a fourth saddle link 723 nested together with pullwires 96a, 96b, 96c passing through the walls of the links and terminating in the first saddle link 720. By applying tension to pullwire 96c in the direction of arrow 730, the plurality of links 720, 721, 722, 723 rotate along their mated surfaces 706, 708 in the direction of the flanges 704 toward pullwire 96c, as shown. This steers the shaft 20 of the main body 10 in a first direction. By applying tension to pullwire 96a, the plurality of links 720, 721, 722, 723 rotate along their mated surfaces 706, 708 in the direction of the flanges 704 toward pullwire 96a. This steers the shaft 20 of the main body 10 in a second direction, opposite the first direction. Applying tension to pullwire 96b does not steer the shaft 20 in a new direction since the pullwire 96b does not pass through a flange 704. However, applying such tension may assist in locking the plurality of links 720, 721, 722, 723 in a desired configuration. Thus, saddle links 700 allow portions of the main body 10 to be steered in two directions, such as in a wagging motion. Furthermore, saddlelinks 700 provide a nested link that may be steered and/or shape-locked, and that also effectively transmits torque. In this manner, saddle links 700 provide many of the benefits of pinned nested links without requiring pins.

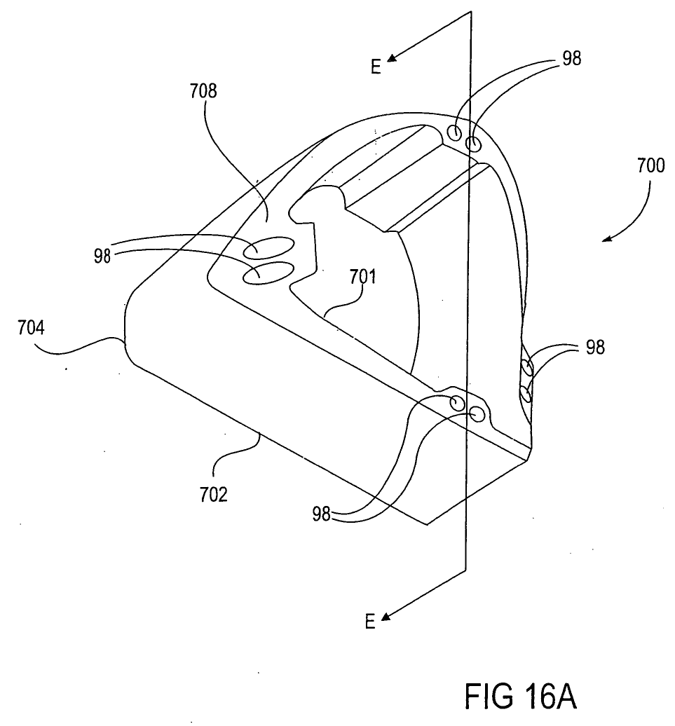

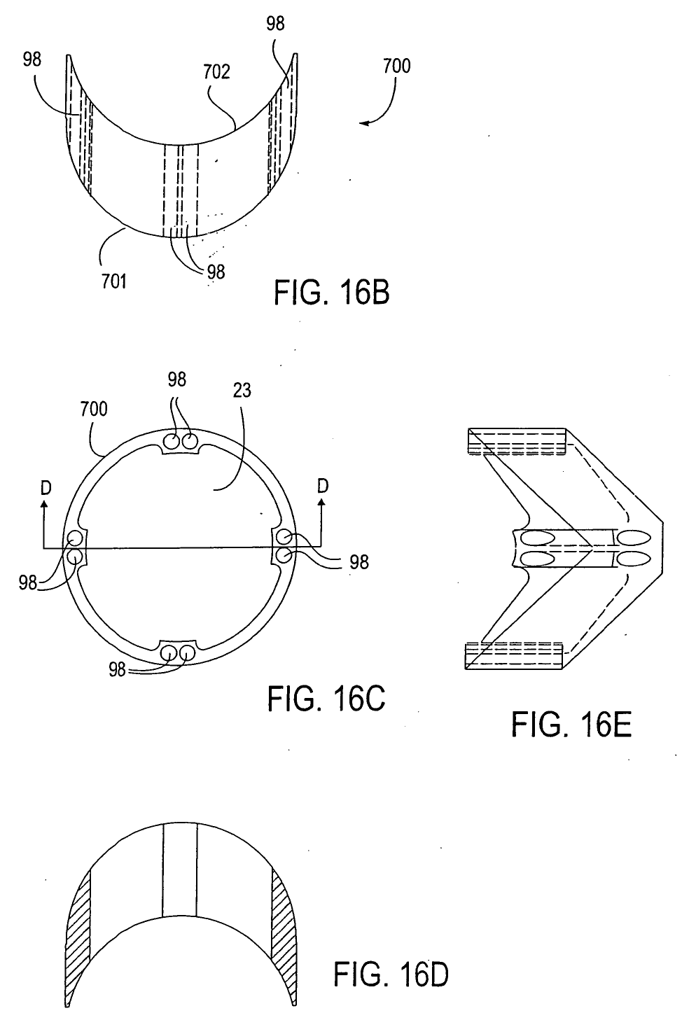

FIGS. 16A-16E illustrate another embodiment of a saddle link 700. FIG. 16A provides a perspective view showing a top edge 701 and a bottom edge 702. Here, the saddle link 700 includes two flanges 704 (one shown, one hidden), each flange 704 extending in the same direction on opposite sides of the saddle link 700 forming a curvature of the bottom edge 702. Beveling of the inside of the link 700 along the bottom edge 702 forms a bottom mating surface 706 (hidden). The top edge 701 follows a curvature which is similar yet less dramatic than the bottom edge 702. Beveling of the outside of the link 700 along the top edge 701 forms a top mating surface 708. In addition, pullwire lumens 98 are shown extending from the top mating surface 708 to the bottom mating surface 706 through each flange 704. In addition, pullwire lumens 98 are present extending through the wall of the link 700 at locations equidistant between the flanges 704. FIG. 16B provides a side view of the saddle link 700 of FIG. 16A, and FIG. 15C provides a bottom view of the saddle link 700 of FIG. 16A. FIG. 16D provides a cross-sectional view along D-D of FIG. 16C, and FIG. 16E provides a cross-sectional view along E-E of FIG. 16A.

Rattlesnake Links

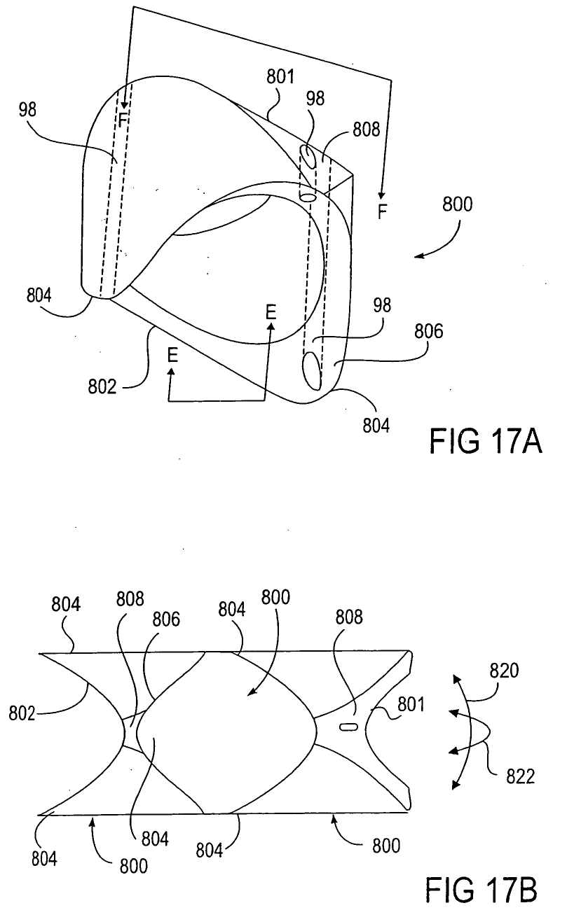

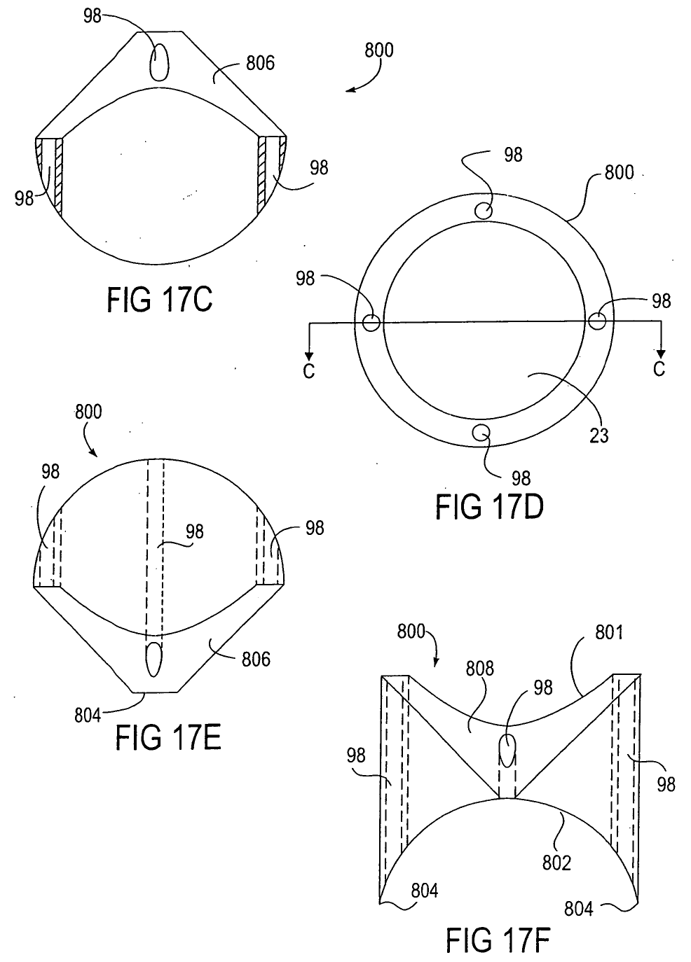

In some embodiments, one or more of the sections 90, 92, 93 of the shaft 20 of the main body 10 comprise a multiplicity of rattlesnake links. FIG. 17A provides a perspective view of an embodiment of a rattlesnake link 800 having a top edge 801 and a bottom edge 802. Here, the rattlesnake link 800 includes two flanges 804, each flange 804 extending in the same direction on opposite sides of the rattlesnake link 800 forming a curvature of the bottom edge 702. Beveling of the inside of the link 800 along the bottom edge 802 forms a bottom mating surface 806. The top edge 801 follows a curvature which is opposite to the bottom edge 802, the top edge 801 inversely mimics the bottom edge 802 yet in a less dramatic fashion. Beveling of the outside of the link 800 along the top edge 801 forms a top mating surface 808. In addition, pullwire lumens 98 are shown extending from the top mating surface 808 to the bottom mating surface 806 through each flange 804. In addition, pullwire lumens 98 are present extending through the wall of the link 700 at locations equidistant between the flanges 804.

Stacking of a plurality of rattlesnake links 800 is shown in FIG. 17B. Here it can be seen that the links 800 are disposed so that a bottom mating surface 806 of link 800 coacts with a top mating surface 808 of an adjacent link. Since the top edge 801 of each link 800 inversely mimics its bottom edge 802, the links 800 are stacked in alternating fashion, with each link 800 offset 90 degrees from an adjacent link. This creates a “rattlesnake”-like appearance to the shaft 20 of the main body 10. Generally, the adjacent surfaces 806, 808 are contoured to mate so that surfaces 806, 808 can rotate relative to one another. The flanges 804 allow rotation of the links 800 in the direction of the flanges 804. Since the position of the flanges 804 alternate with each link 800, the shaft 20 is able to be steered in four directions, along arrow 820 and arrow 822 in FIG. 17B. This arrangement links 800 allows steering in a variety of directions while providing torque control and enhanced flexibility, as compared to the saddle links described previously.

FIGS. 17C-17F illustrate additional views of the embodiment of the rattlesnake link 800 of FIG. 17A. FIG. 17C provides a cross-sectional view along C-C of FIG. 17D which provides a bottom view of the link 800 of FIG. 17A. FIG. 17E provides a cross-sectional view along E-E of FIG. 17A, and FIG. 17F provides a cross-sectional view along F-F of FIG. 17A.

### EXAMPLES

As stated previously, the above embodiments may be used to construct any or all of the sections 90, 92, 93. Furthermore, additional and/or alternative sections may be provided. A few examples are provided herein to illustrate this feature.

In a first example, the main body 10 has a first section 90 comprised of nested links 260, a second section 92 comprised of bump links 62 and a third section 93 comprised of pin links 160. As described, the first section 90 is the most proximal section and typically involves the least amount of articulation. Therefore, the nested links 260 provide basic articulation and optional shape-locking. The second section 92 typically involves retroflexion and optional shape-locking. As described previously, bump links 62 are well suited for retroflexion since the links 62 may be designed to steer the section 92 into a predetermined arrangement. The links 62 are pivotally connected by hinge structures 100 and gaps 102 are present on opposite sides of the structures 100. During retroflexion, the section 92 curves until the gaps 102 close forming the desired retroflexed curvature. The third section 93 typically involves articulation in a single plane, such as a wagging motion, or in more than one plane, such as 360 degree motion. The pin links 160 are well suited for such articulation since the links 160 may be articulated in any direction. In addition, the pin links 160 have a torque transmitting feature that is particularly suitable for the third section 93 which is the most distal section.

In a second example, the main body 10 has a first section 90 and a second section 92 comprised of nested links 260, and a third section 93 comprised of pin links 160. The nested links 260 provide basic articulation and optional shape-locking which are suitable for articulation of the first section 90 and retroflexion of the second section 92. Also, it may be appreciated that the second section 92 may be articulated without retroflexion. The pin links 160 are well suited for the third section 93 as described above.

In a third example, the main body 10 has a first section 90 comprised of a flexible or semi-flexible tube, a second section 92 comprised of nested links 260, and a third section 93 comprised of pin links 160. As previously mentioned, the flexible or semi-flexible tube of the first section 90 may be comprised of a solid plastic or polymeric material, such as polyurethane, nylon or polyvinyl chloride (PVC). This allows deflection when only minimal passive steering capabilities by this section are desired. Again, the bump links 262 provide retroflexion and optional shape-locking which are suitable for the second section 92 while the pin links 160 are well suited for the third section 93 as described above.

IV. Side Opening

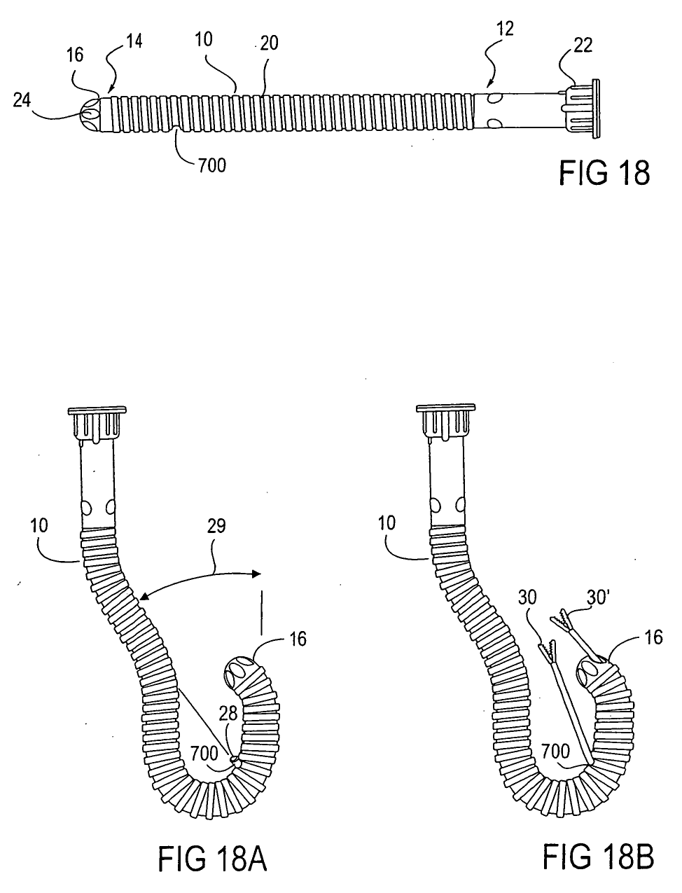

FIG. 18 illustrates another embodiment of the main body 10. Here the main body 10 includes a side opening 700. The side opening 700 leads to an internal lumen within the main body 10 so that an instrument may be passed through this internal lumen exiting the main body 10 through the side opening 700 rather than through an opening at the distal tip 16. The internal lumen may be any lumen previously described, such as the central lumen 23, scope lumen 24, arm guide lumen 26, insufflation lumen 56, auxiliary lumen 58, or irrigation/suction lumen 60, or an additional lumen. Any type of instrument may be passed through the internal lumen and may be extended any distance from the side opening 700. FIG. 18A illustrates a scope 28 protruding from the side opening 700. Typically, the field of view (indicated by arrow 29) of the scope 28 spans up to approximately 140 degrees. This may allow the visualization of the distal tip 16 and surrounding area to facilitate desired steering of the distal tip 16 and/or any procedures performed in the surrounding area. FIG. 18B illustrates a tool arm 30 protruding from the side opening 700. The tool arm 30 may extend any distance, such as near the distal tip 16 to function in coordination with a tool arm 30′ protruding from an opening in the distal tip 16.

It may be appreciated that the side opening 700 may be used to administer or remove liquids, gases, or other substances, or to create a vacuum, to name a few. Further, the side opening may be connected with a lumen which is external to the main body 10, such as a lumen through a tubing attached to the outside of the shaft 20 of the main body 10. It may also be appreciated that the main body 10 may include more than one side opening 700 and/or the side opening 700 may be present without any openings at the distal tip 16.

If multiple side openings 700 are provided, multiple tools, scopes, etc. may be advanced therethrough and used in a coordinated fashion, or may be used independently. For example, two scopes may be provided and used in coordinated fashion to provide stereoscopic or three-dimensional visualization. Additionally or alternatively, the scopes may be used independently to provide multiple vantage points. It should be understood that multiple scopes alternatively may be advanced through, or provided near, distal tip 16 of main body 10 for coordinated or independent use.

V. Handle

It may be appreciated that the handle 22 of the present invention may have any suitable form. The handle 22 may provide a variety of functions, including but not limited to controlling tension in the pullwires 96 or tension wires. Tension control includes applying tension to various pullwires to steer specific portions of the main body or applying a proximal force to one or more of the pullwires to lock specific portions of the main body in a desired shape. Tension control may be provided with a variety of mechanisms, including pulleys, spools, knobs, ratchets, and gears, to name a few. In some embodiments, separate steering and locking mechanisms are present to control each section of the main body, such as sections A, B, and C. In other embodiments, a steering or locking mechanism may control more than one section of the main body.

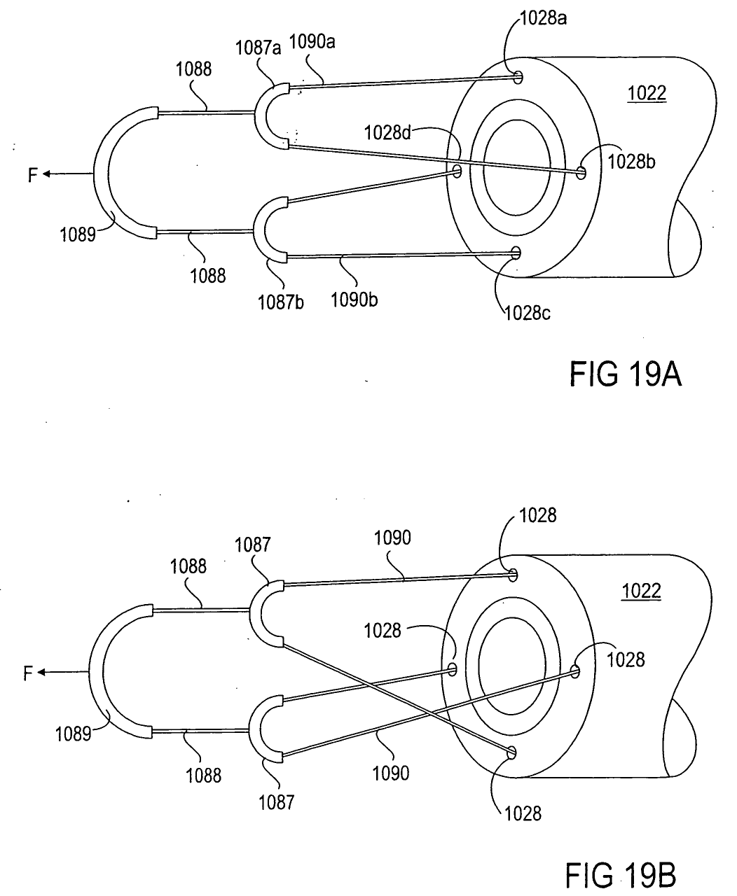

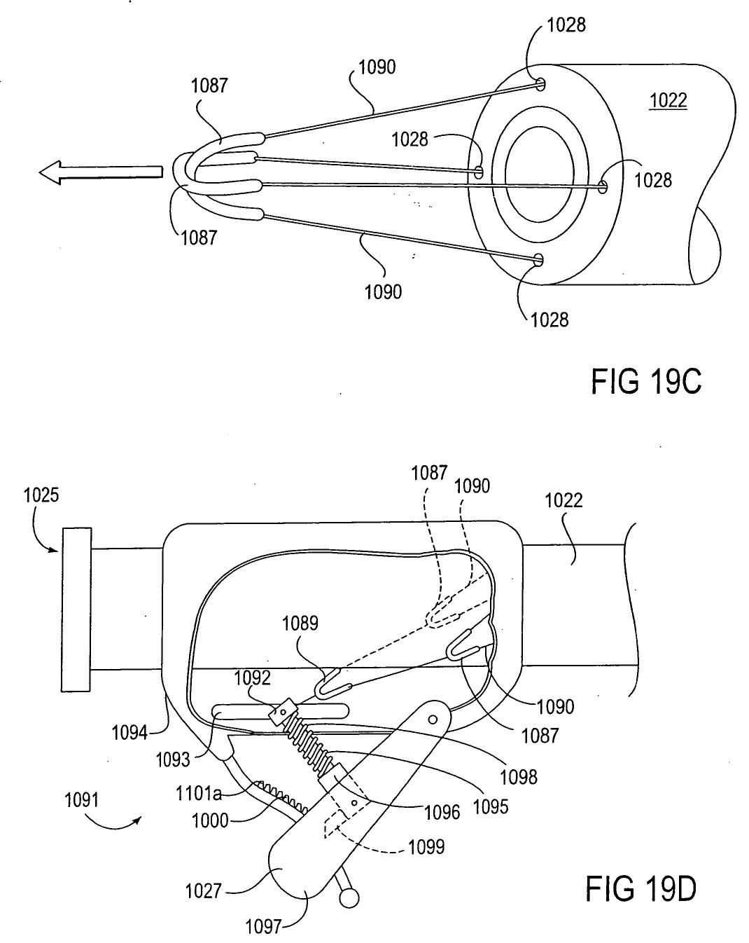

Some examples of tension control mechanisms are provided in U.S. patent application Ser. No. 10/281,462, filed on Oct. 25, 2002, which is incorporated herein by reference for all purposes. FIG. 19A schematically depicts components of a first embodiment of a tensioning mechanism having plurality of distal pulleys 1087a, 1087b operably coupled via proximal tension wire 1088. Proximal tension wire 1088 is slidably disposed within proximal pulley 1089. Each tension wire 1090a couples adjacent tension wire lumens 1028, through respective distal pulleys 1087. For example, if four tension wire lumens 1028a-1028d are provided, as in FIG. 19A, first tension wire 1090a extends from tension wire lumen 1028a to adjacent tension wire lumen 1028b through first distal pulley 1087a. Likewise, second tension wire 1090b extends from tension wire lumen 1028c to adjacent tension wire lumen 1028d through second distal pulley 1087b.

This configuration equalizes tension within tension wires 1090, so that a proximally directed force F applied to proximal pulley 1089 is distributed evenly through tension wires 1090. When one of the tension wires breaks, this configuration allows overtube 1022 or main body section to soften into its flexible state since the loss of tension in any of the tension wires is transmitted through the pulley system to the remaining tension wires.

It will be apparent to one of ordinary skill in the art that tension wires 1090a and 1090b may comprise either two separate lengths of wire, or a single length of wire that is looped backwards after traversing the distal-most nestable element or linkage. Furthermore, while FIG. 19A depicts tension wires 1090 extending through adjacent tension wire lumens 1028, the tension wires instead may extend through wire lumens disposed diametrically opposite each other, as shown in FIG. 19B. Tension wires 1090 preferably are made from a superelastic material, e.g., nickel titanium alloy, but also may be made from braided stainless steel, single stainless steel wires, Kevlar, a high tensile strength monofilament thread, or combinations thereof. These materials are provided only for the sake of illustration and should in no way be construed as limiting.

In an alternative embodiment illustrated in FIG. 19C, proximal pulley 1089 is eliminated, and distal pulleys 1087 are fixed to each other, e.g., by welding, so that a unitary pulley manifold is formed. A proximally directed force F that is applied to the pulley manifold is distributed evenly through tension wires 1090 that extend through respective distal pulleys 1087 to diametrically disposed tension wire lumens 1028 within overtube 1022. If tension wires 1090 comprise two separate lengths of wires, the risk of reconfiguration of the main body portion or overtube 1022 is reduced if one of the wires breaks since the tension within the overtube, as defined by the unbroken tension wire, is symmetrically balanced. The portion of the main body also may comprise only one distal pulley 1087 coupled to overtube 1022 via a single tension wire 1090 disposed through diametrically opposite tension wire lumens 1028. When a proximally directed force is applied to the single distal pulley, the force is distributed through the single tension wire to impose a symmetrical compressive clamping load on overtube 1022 that is sufficient to shape-lock the overtube.

Referring now to FIG. 19D, an embodiment of a locking mechanism within a handle 1091 is depicted. In this embodiment, tension wires 1090 extend from a portion of the main body or overtube 1022 to handle 1091. Within lumen 1025, the tension wires are slidably coupled to distal pulleys 1087, which in turn are slidably coupled to proximal pulley 1089. Proximal pulley 1089 is coupled to and translates with slide block 1092, that is keyed to travel along track 1093 disposed within housing 1094. Plunger 1095 is mounted pivotally to slide block 1092 at the proximal end and slidably disposed within plunger housing at a distal end.

Plunger housing 1096 is mounted pivotally to actuator 1027, illustratively hand grip 1097. To bias hand grip 1097 against actuation absent an externally applied force, compression spring 1098 is provided concentrically disposed about plunger 1095. Compression spring 1098 maintains tension wires 1090 in constant tension when the tensioning mechanism is actuated to impose a clamping load. Advantageously, if adjacent nestable elements shift slightly when overtube 1022 is shape-locked, the proximal bias of compression spring 1098 immediately advances slide block 1092 in the proximal direction to maintain a relatively constant tension load within tension wires 1090, thereby reducing the risk of reconfiguration of the overtube back to the flexible state that otherwise may occur absent compression spring 1098.

Hand grip 1027 also includes pawl 1099, which is disposed to engage teeth 1000 on ratchet bar 1101 to prevent distally-directed motion of slide block 1092. Ratchet bar 1101 is pivotally mounted in housing 1094 with a spring (not shown) that, with the aid of compression spring 1098, biases pawl 1099 against teeth 1000 of ratchet bar 1101, to provide a one-way ratchet effect when hand grip 1097 is squeezed.

In operation, squeezing hand grip 1097 causes pawl 1099 to capture the next proximal-most tooth 1000. This movement also provides a compressive force to compression spring 1098 that is transmitted to slide block 1092. The proximally-directed component of the compressive force causes slide block 1092 to translate along track 1093, proximally retracting tension wires 1090 so that a clamping load is imposed on the nestable elements within the portion of the main body or overtube 1022 being locked.

Advantageously, proximal-most tooth 1000a is disposed on ratchet bar 1101 at a predetermined proximal location that permits a single actuation of hand grip 1097 to completely transition the desired portion of the main body or overtube 1022 from a flexible state to a locked or shape-fixed state. Furthermore, as pawl 1099 advances hand grip 1097 closer to housing 1094, the mechanical advantage of the actuation of the hand grip increases. More specifically, as hand grip 1097 becomes increasingly horizontal, the proximally-directed component of the force transmitted by compression spring 1098 increases in magnitude. Accordingly, more force is transmitted to increase tension within tension wires 1090, and thus increase the clamping load applied to rigidize the portion of the overtube 1022.

When it is desired to transition the portion of the overtube 1022 into the flexible state, pawl 1099 is released from engagement with teeth 1000 by rotating ratchet bar 1101 in the proximal direction. The release of the compressive load applied to compression spring 1098 causes hand grip 1097 to rotate in the distal direction and slide block 1092 to retract in the distal direction. This sufficiently relaxes tension wires 1090 so that the tension wires retain little to no tension, thereby permitting overtube 1022 to assume its most flexible state.

It may be appreciated that the locking mechanism illustrated in FIG. 19D may be used to lock the entire length of the main body in a desired shape, or may lock a specific portion of the main body. Likewise, the handle may include a plurality of locking mechanisms, each used to lock a different portion of the main body. In addition, the handle may include steering mechanism which are used to independently steer different portions of the main body. In addition, a steering mechanism may include separate mechanisms for rough steering and fine steering. For example, the steering mechanism may include a knob for rough steering wherein turning of the knob applies tension to a pullwire by wrapping a portion of the pullwire around a spool. The steering mechanism also may include a wheel for fine steering wherein rotation of the wheel applies tension to a pullwire by the engagement of gears. Such examples are illustrative and it may be appreciated that any suitable steering mechanism may be used.

VI. Bite Block Connections

As mentioned, the main body 10 of the present invention may be used to access the upper gastrointestinal tract and/or stomach through the esophagus. For example, FIGS. 8F-8H illustrate positioning of an embodiment of the main body 10 within a stomach S through an esophagus E. Typically, at least a portion the main body 10 remains within or protrudes from the patient's mouth throughout the procedure. To assist in retaining the main body 10 inside the mouth so as to prevent bite damage, the main body 10 may include a bite block.

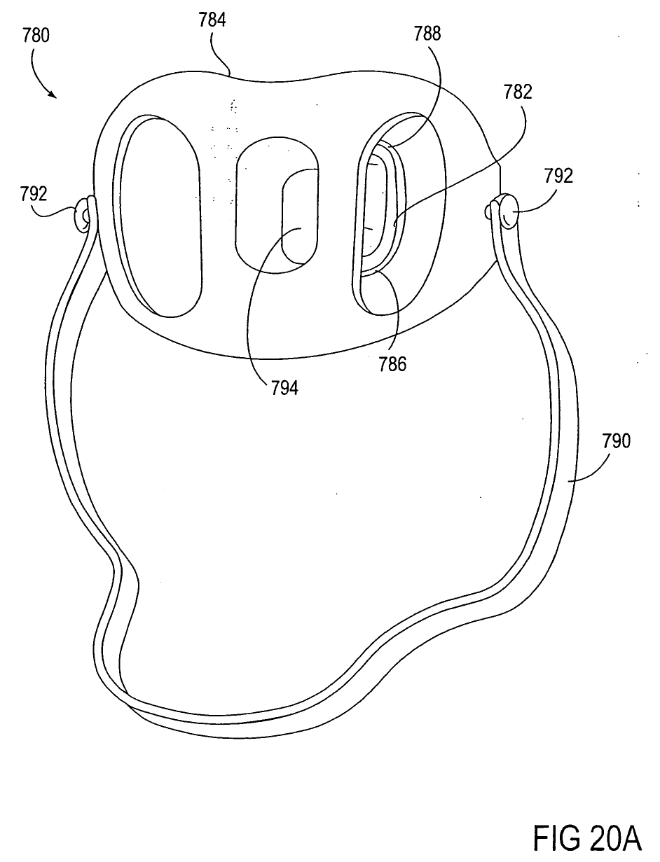

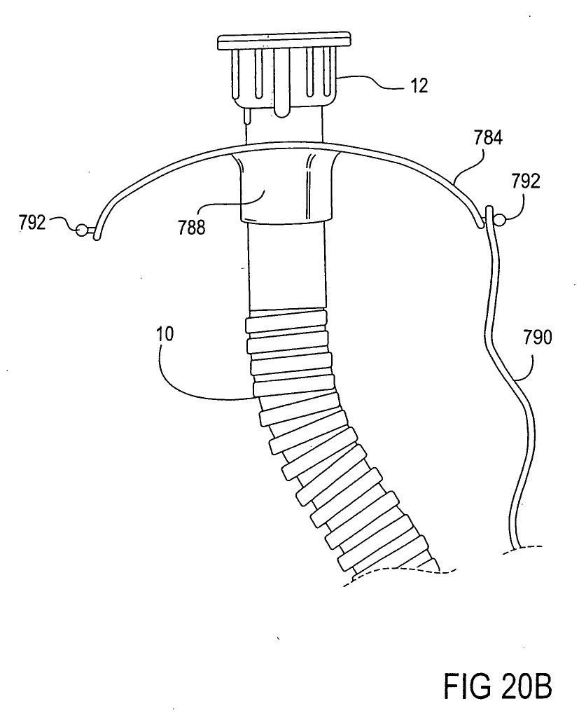

FIG. 20A illustrates an embodiment of a bite block 780 which may be used with the present invention. In this embodiment, the bite block 780 includes a mouthpiece 784, configured for insertion into a patient's mouth, which is attached to a facepiece 786, configured to remain outside of the patient's mouth. The mouthpiece 784 includes a bottom tongue pad 786 which is positionable against the tongue and an upper part 788 which is positionable under the upper teeth. The bite block 780 also includes a strap 790 which is fixedly attached to the facepiece 786 or attachable to hooks or T-bars 792 on the facepiece 786. The strap 790 extends around the head of the patient to hold the bite block 780 in place. The bite block 780 includes an orifice 794 through which the main body 10 is passable. FIG. 20B illustrates the bite block 780 having an embodiment of the main body 10 passed therethrough. Thus, the proximal end 12 of the main body 10 remains outside of the patient's mouth. In this arrangement, the bite block 780 holds the main body 10 in a stable position within the patient's mouth to prevent any bite damage.

It may be appreciated that the bite block 780 may be fixedly attached to or integral with the main body 10. Or, the bite block 780 may be separate and mountable on the main body 10. Thus, any suitable commercially available bite block 780 may be used, such as UltimaBloc® provided by GI Supply (Camp Hill, Pa.).

VII Suction and Suction Plication

As mentioned previously, the main body 10 may have a single lumen therethrough for a variety of uses, such as for passage of one or more instruments or for the passage of air or fluid, such as for aspiration or suction. Similarly, the main body 10 may have more than one lumen passing therethrough, each lumen used for a different function. In some embodiments, one or more lumens in the main body 10 are used for suction. Suction may be useful in a variety of procedures such as gastrointestinal cleaning of fluid, biomatter or blood or for degassing, to name a few. Further, suction may be used in plication procedures, as will be described in more detail below. Examples of suction and plication systems and procedures for their use are provided herein and in copending U.S. patent Ser. No. 10/735,030, incorporated herein by reference for all purposes.

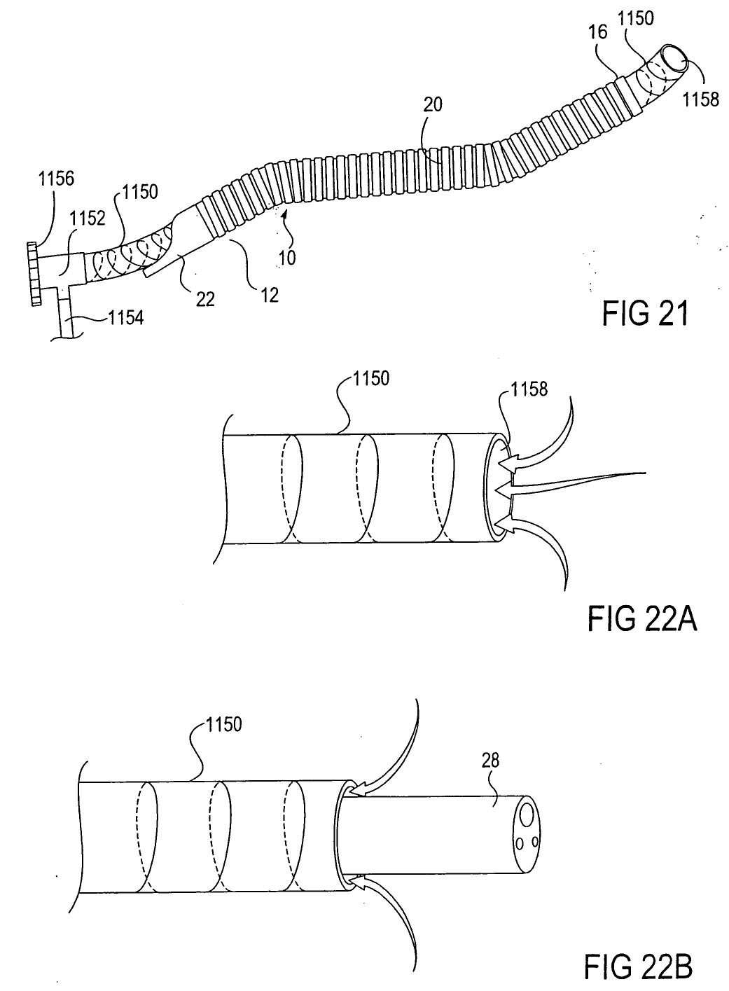

FIG. 21 illustrates an embodiment of the main body 10 having a deflectable and/or steerable shaft 20. It may be appreciated that the main body 10 may have links, hinges, coils or other similar structures to allow deflection in one or more sections as in any of the embodiments described above. As shown, a handle 22 is disposed near the proximal end 12 of the main body 10, e.g. to steer or shape-lock the shaft 20 in one or more curved positions. In this embodiment, a tubing 1150 passes through the handle 22 and the main body 10 near or beyond the distal tip 16. Typically, the tubing 1150 is spring reinforced to prevent collapse of the tubing 1150 during suction, however the tubing 1150 may be comprised of any suitable non-collapsing material. A Y-fitting 1152 joins the tubing 1150 with a suction line 1154 and provides a fitting 1156 for the insertion of tools or instruments through the main body 10. In this embodiment, the tubing 1150 has a main lumen 1158 therethrough.

As shown in FIG. 22A, suction can be applied to the main lumen 1158 to draw fluids or gases into the tubing 1150, as indicated by arrows. Similarly, as shown in FIG. 22B, a scope 28 may be advanced through tubing 1150 and suction applied around the scope 28 to draw fluids or gases into the tubing 1150, as indicated by arrows. It may be appreciated that other instruments may be present instead of or in addition to the scope with suction applied therearound.

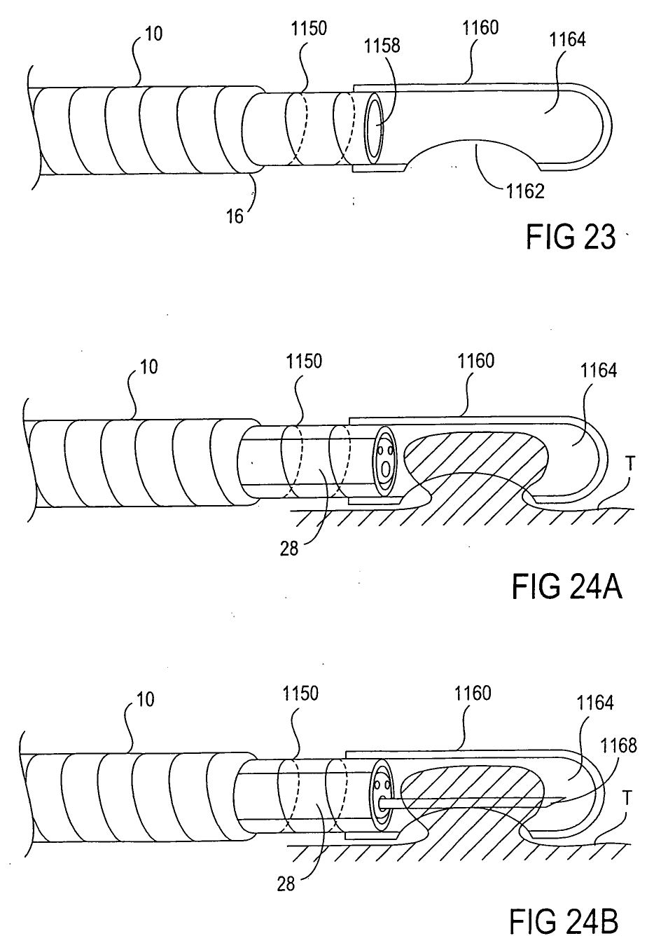

Further, as illustrated in FIG. 23, a suction cap 1160 may be joined with or attached to the tubing 1150. The suction cap 1160 has a port 1162; in this example the port 1162 is disposed on the side of the suction cap 1160 to create a cavity 1164 within the cap 1160. The suction cap 1160 may be used to apply suction for evacuation or cleaning of a cavity or area within the body. Or, as shown in FIG. 24A, when the port 1162 is positioned near tissue T and suction is applied, tissue T can be drawn through the port 1162 into the cavity 1164, for example, to plicate the tissue. Passage of a scope 28 through the tubing 1150 allows viewing of the tissue T, as shown.

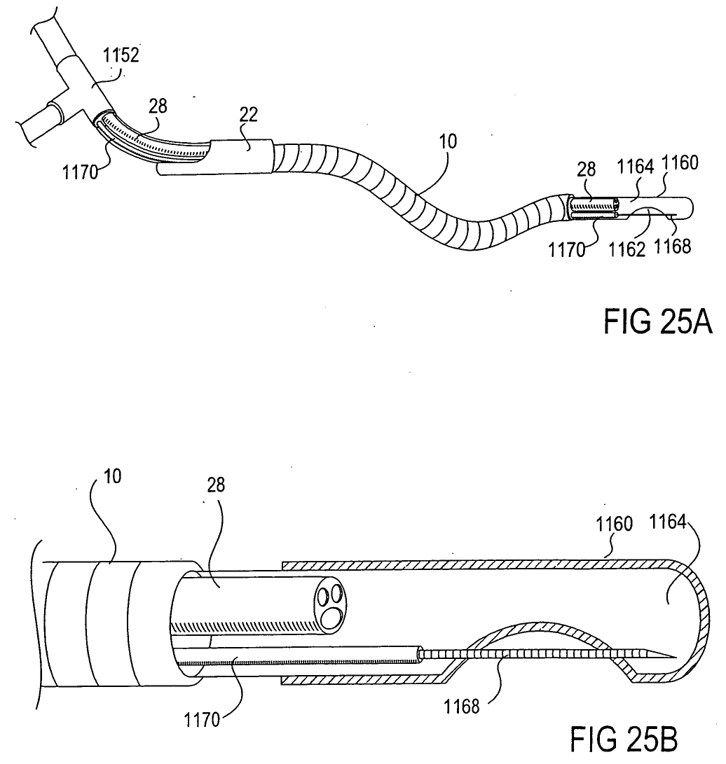

In addition, an instrument may be passed through the scope 28 or through the tubing 1150 to manipulate the suctioned tissue T. For example, FIG. 24B illustrates a needle 1168 passing through the scope 28 and piercing the tissue T. Although FIG. 24B illustrates a needle 1168 passing through the scope 28, the needle 1168 or other instrument may alternatively be passed through the main body 10 separately from the scope 28. For example, FIG. 25A illustrates a scope 28 passing through the main body 10 and an instrument guide 1170 also passing through the main body 10. The scope 28 and instrument guide 1170 both extend to the suction cap 1160, though they may move independently or may be fixed in place. An instrument, such as a needle 1168, may be passed through the instrument guide 1170, such as shown in FIG. 25A. The scope 28 and guide 1170 may be positioned within the main body 10 in any desired arrangement, particularly wherein the guide 1170 is directed toward the port 1162 and the scope 28 is directed to visualize the cavity 1164 near the port 1162, as shown in FIGS. 25A-25B. FIG. 25B provides an enlarged view of the suction cap 1160 of FIG. 25A. Here, a needle 1168 is shown extending through the guide 1170 and into the cavity 1164. Thus, any tissue suctioned though the port 1162 would be pierced by the needle 1168 and would be observable through the scope 28.

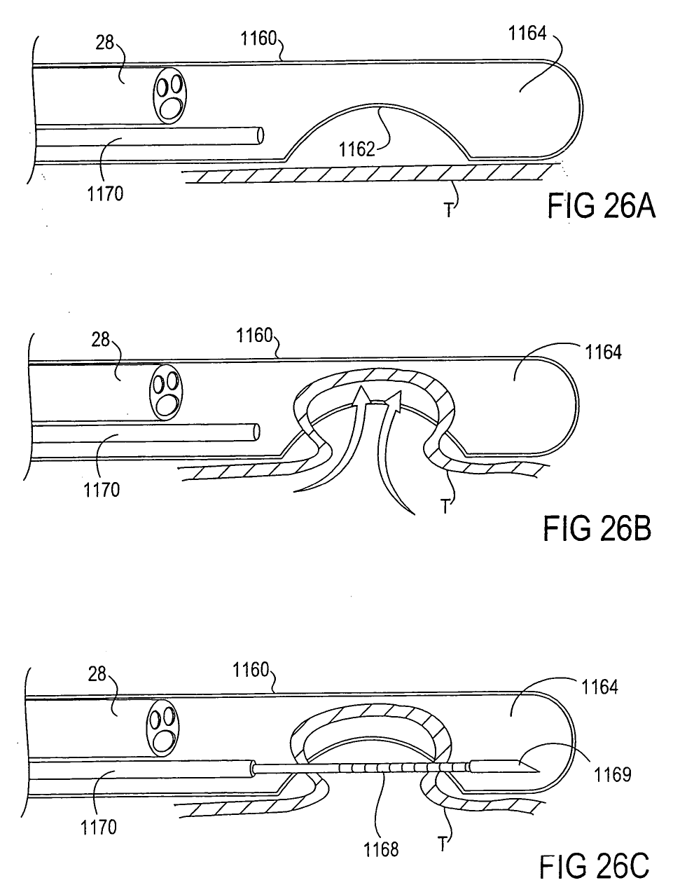

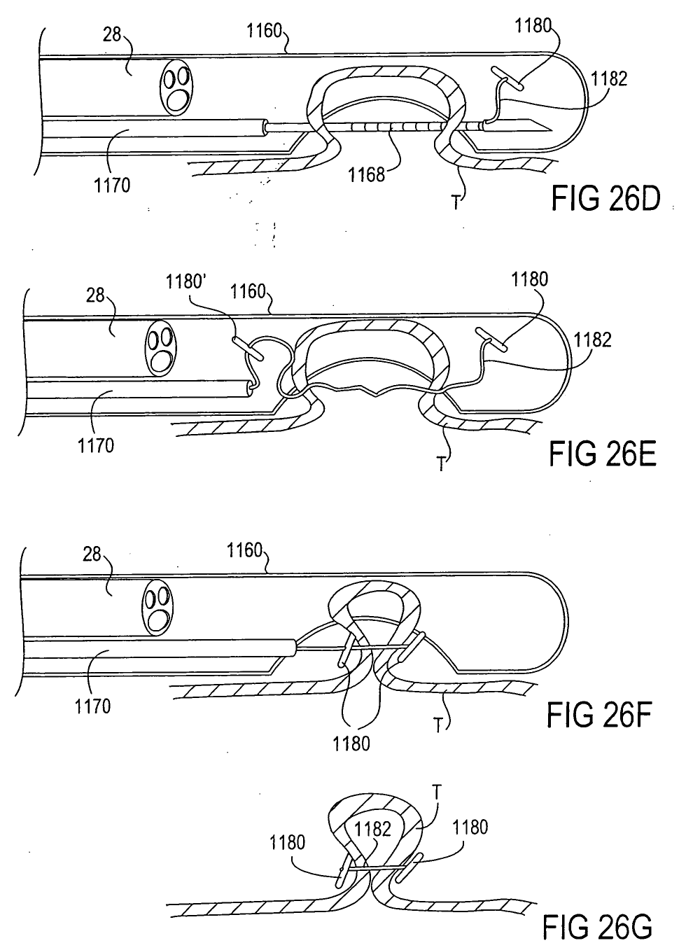

FIGS. 26A-26G illustrate an exemplary method of plicating tissue using apparatus of the present invention. FIG. 26A illustrates a suction cap 1160 forming a cavity 1164 and having a port 1162. A scope 28 and an instrument guide 1170 extend into the cavity 1164, as shown. The port 1162 is positioned near a tissue T to be plicated. As depicted in FIG. 26B, suction is applied to the cavity 1164 and a portion of the tissue T is pulled up through the port 11162 into the cavity 1164, thereby plicating the tissue. Due to the shape of the suction cap 1160, the suctioned tissue T forms a balloon or bulb. A needle 1168 is then advanced through the instrument guide 1170, piercing one side of the tissue T bulb, traversing the port 1162 and piercing the other side of the tissue T bulb, as illustrated in FIG. 26C. The needle 1168 may have a specialized tip 1169 which assists in piercing and/or prevents coring of the tissue T. In this example, the specialized tip 1169 also houses an anchor 1180 attached to a suture 1182, wherein the anchor 1180 is released on the other side of the tissue T bulb, as shown in FIG. 26D. The needle 1168 is then retracted and another anchor 1180′ attached to the suture 1182 is released on the one side of the tissue T bulb as shown in FIG. 26E. These steps may be observed through scope 28 directed toward the cavity 1164. The suture 1182 is then cinched, as shown in FIG. 26F, to draw the anchors 1180, 1180′ together at the base of the tissue T bulb, thereby securing and maintaining the plicated tissue T. The suction cap 1160 is then removed leaving the tissue T plicated by the anchors 1180, 1180′, as shown in FIG. 26G.

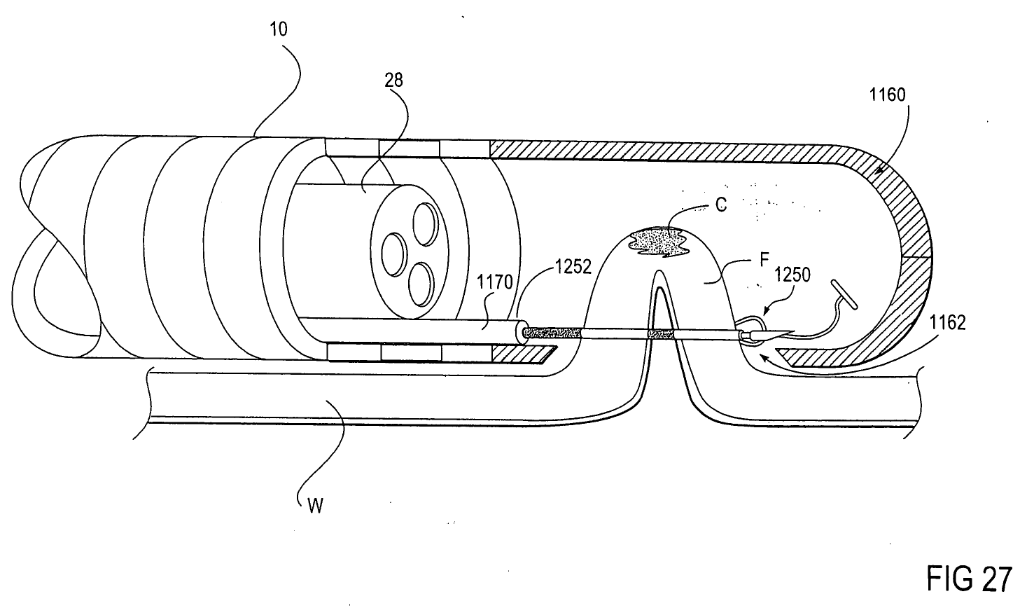

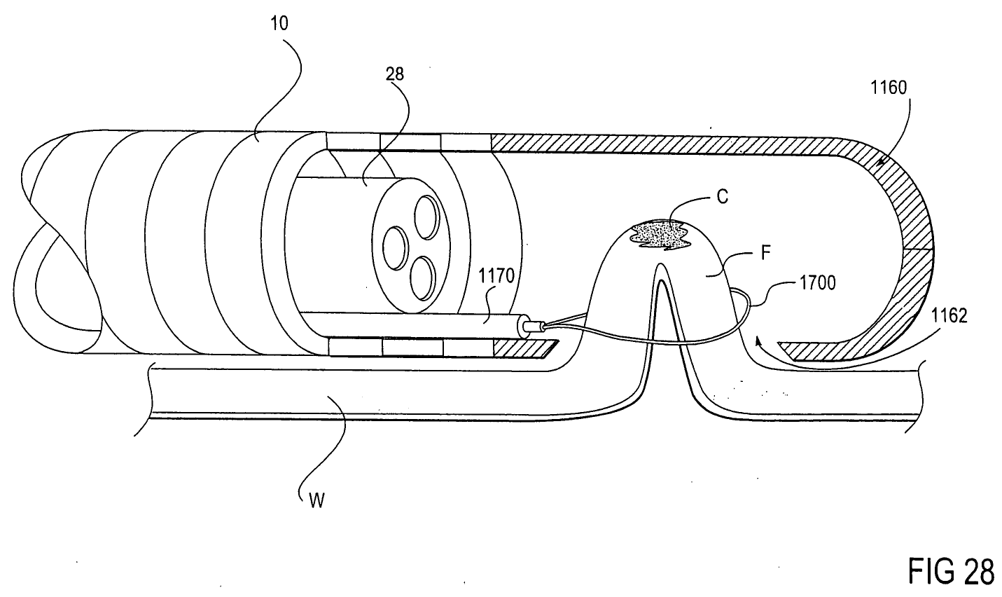

With reference to FIG. 27, an exemplary method of resecting a lesion or early cancer C, e.g. within a patient's gastrointestinal tract, using apparatus of the present invention is described. In FIG. 27, a shape-lockable main body 10 having a suction cap 1160 coupled to its distal end is shown. A scope 28 and instrument guide 1170 is shown disposed within the suction cap 1160, extending from the main body 10. Instrument guide 1170 optionally may comprise delivery tube 1252 of anchor delivery system 1250, as described hereinbelow with respect to FIG. 28, or may comprise the delivery tube of any alternative anchor delivery system described, such as described in copending U.S. patent Ser. No. 10/735,030, filed Dec. 12, 2003, incorporated herein by reference for all purposes. Furthermore, guide 1170 may be coupled to main body 10 or may be advanceable relative to the main body 10.

As shown, the suction cap 1160 comprises port 1162 to facilitate side-suction plication of tissue. Suction cap 1160 additionally or alternatively may comprise one or more apertures at its distal end (not shown) to facilitate end-suction plication of tissue. Suction cap 1160 and the main body 10 preferably are sealed along their lengths, such that suction may be drawn through the main body 10 and suction cap, e.g., via a suction pump (not shown) coupled to a proximal region of main body 10 external to the patient.

Advantageously, as compared to previously-known suction plication apparatus, steerable and/or shape-lockable main body 10 allows system of tools, such as the suction plication system, to be positioned at, or steered to, a treatment site while the main body 10 is disposed in a flexible state. Main body 10 then optionally may be transitioned to a rigid state prior to drawing of suction through suction cap 1160. In this manner, the suction cap 1160 and associated instruments used in suction plication may be directed to, and maintained at, a treatment site during a medical procedure.

In FIG. 28, main body 10 has been endoscopically advanced, e.g., through a patient's esophagus or colon, under endoscopic visualization provided by scope 28, to a vicinity of lesion or early cancer C along tissue wall W, while the main body 10 was disposed, in a flexible state. Main body 10 alternatively may be advanced laparascopically, e.g. through a trocar. The main body 10 preferably is then transitioned to a rigid state in a configuration enabling access, e.g. luminal access, to the lesion or early cancer.

Suction is drawn through main body 10 and suction cap 1160 to urge tissue in the vicinity of lesion/early cancer C through side port 1162 and into cavity 1164 thereby forming tissue fold F. As can be verified by endoscopic visualization, lesion or cancer C resides on the folded tissue. The lesion, polyp, cancer, etc. then may be removed via cutting apparatus, such as snare or resection loop 1700 advanced through instrument guide 1170. As will be apparent to those of skill in the art, alternative plication apparatus in accordance with the present invention may be used to resect lesion C.

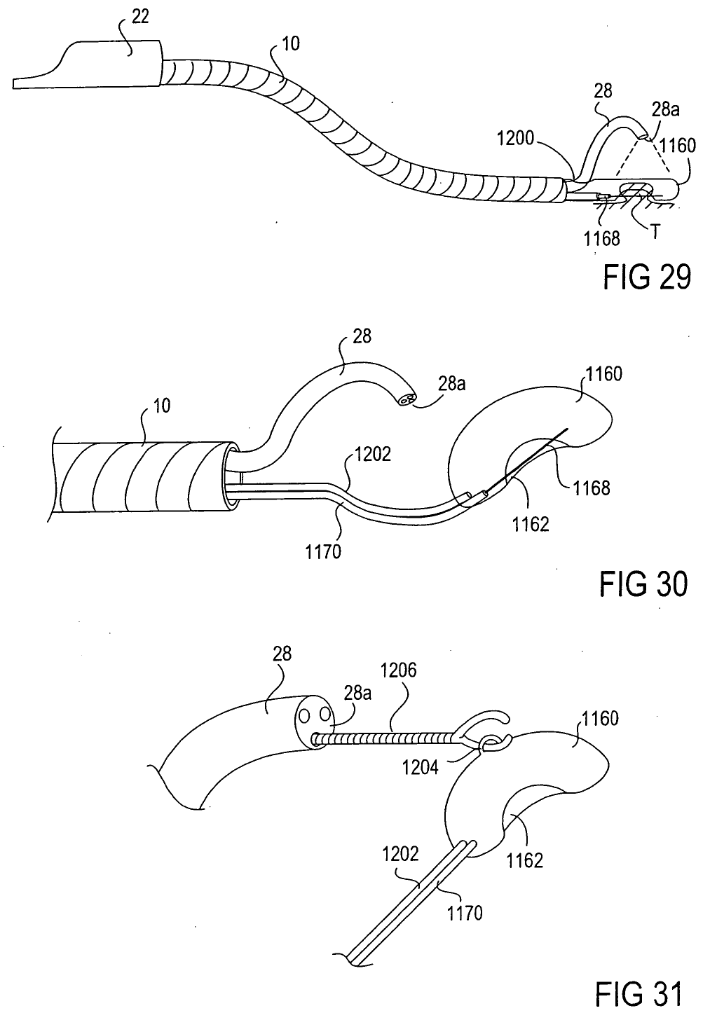

In the above embodiments, the scope 28 was aligned axially within the suction cap 1160 for visualization of the suction and optionally plication procedure. However, in other embodiments, the scope 28 may be steered to view the procedure from a perpendicular or other angled view. For example, as shown in FIG. 29, the suction cap 1160 may include an exit port 1200 through which the scope 28 is able to pass. The exit port 1200 includes a seal around the scope 28 to ensure sufficient suction or evacuation pressure within the suction cap 1160. In this embodiment, the suction cap 1160 is comprised of a transparent material to allow visualization through the cap 1160. As shown, the scope 28 may be steered so that its distal end 28a is directed perpendicularly or at an angle to the longitudinal axis of the cap 1160. Thus, when tissue T is drawn into the port 1162, as illustrated in FIG. 29, and pierced by needle 1168, the procedure may be viewed from a variety of angles surrounding the suction cap 1160.

Another embodiment is illustrated in FIG. 30. Here the suction cap 1160 is disposed at the end of a suction tube 1202. The suction tube 1202 extends from the main body 10 and is used to draw suction through the suction cap 1160. In this embodiment, the instrument guide 1170 extends alongside the member 1202 to the suction cap 1160 so that instruments, such as a needle 1168 may be advanced across the port 1162. The suction tube 1202 and/or the instrument guide 1170 may also be used to position the suction cap 1160 in any desired location. Thus, the suction tube 1202 and/or instrument guide 1170 may be steerable, deflectable or preformed, to name a few. The scope 28 extends separately from the main body 10 and may be steered to any angle in relation to the suction cap 1160. Again, the suction cap 1160 is comprised of a transparent material to allow visualization through the cap 1160. As shown, the scope 28 may be steered so that its distal end 28a is directed perpendicularly or at an angle to the longitudinal axis of the cap 1160. Thus, when tissue is drawn into the port 1162 and pierced by needle 1168, the procedure may be viewed from a variety of angles surrounding the suction cap 1160.

In a similar embodiment, illustrated in FIG. 31, the suction cap 1160 includes a grasping feature 1204 disposed on its surface for grasping with an instrument. In this example, the grasping feature 1204 comprises a loop and the instrument comprises a grasper 1206 which is extended through a working lumen in the scope 28. It may be appreciated, however, that the grasping feature 1204 may have any form including, hooks, loops, grooves, bumps, indents, holes, protrusions or the like. Further, the grasper 1206 may have any suitable form and may be extended from the scope 28, main body 10 or may be a separate instrument, to name a few. Grasping feature 1204 allows the grasper 1206 to move the suction cap 1160 to any desired location. Thus, in this embodiment, the suction tube 1202 and/or instrument guide 1170 optionally may have no steering capabilities since the suction cap 1160 may be positioned by the grasper 1206.

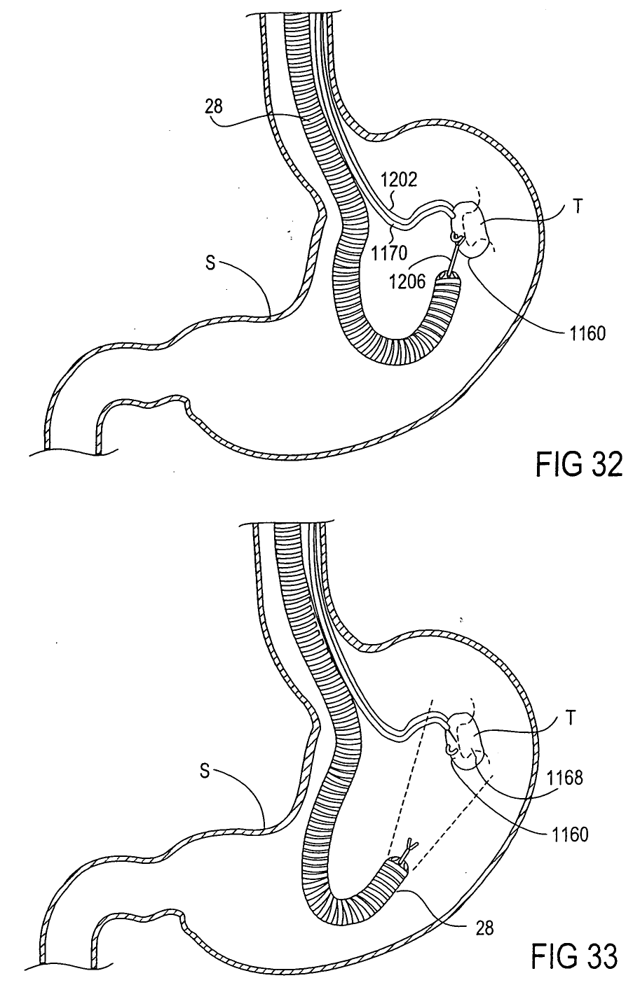

An example of positioning the suction cap 1160 is illustrated in FIGS. 32-33. In FIG. 32, a scope 28 and a suction cap 1160, with an associated suction tube 1202 and instrument guide 1170, are advanced into a stomach S. A grasper 1206 is extended from the scope 28 and grasps a grasping feature on the suction cap 1160. Thus, the suction cap 1160 may be positioned in a desired location within the stomach S by the grasper 1206. In addition, the grasper 1206 may be used to hold the suction cap 1160 in place while suction is applied to tissue T within the stomach S. As shown, tissue T is drawn into the suction tube 1202 during suction. The tissue T may then be pierced, as shown in FIG. 33, by a needle 1168. During the piercing step or at any time during the procedure, the suction cap 1160 may be released from the grasper 1206 and the scope 28 positioned at any location to ensure desired viewing, as illustrated in FIG. 33. The suction cap 1160 may then be regrasped and repositioned at another location within the stomach S.

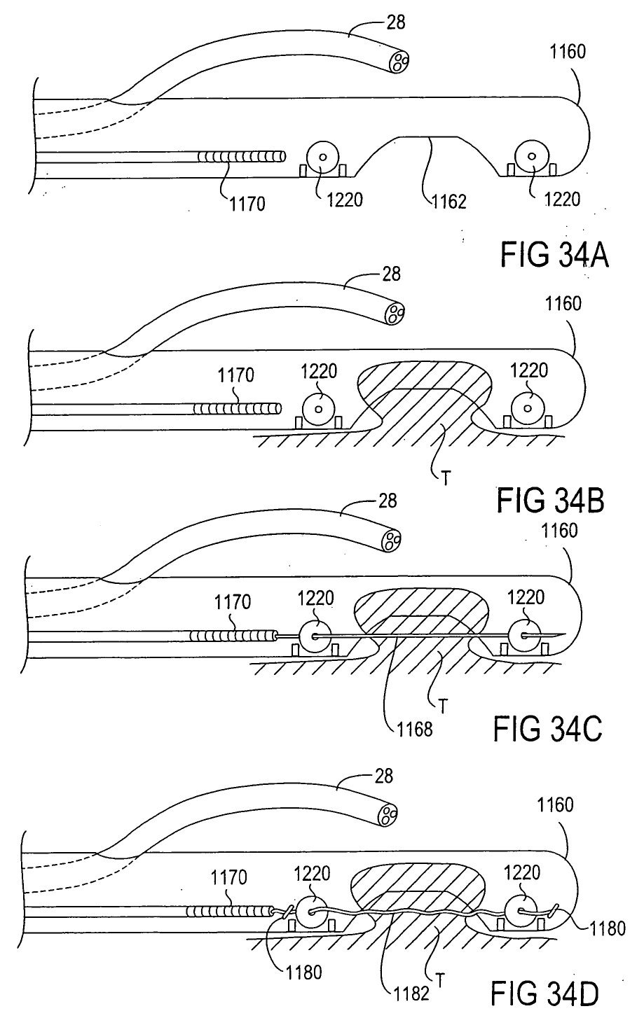

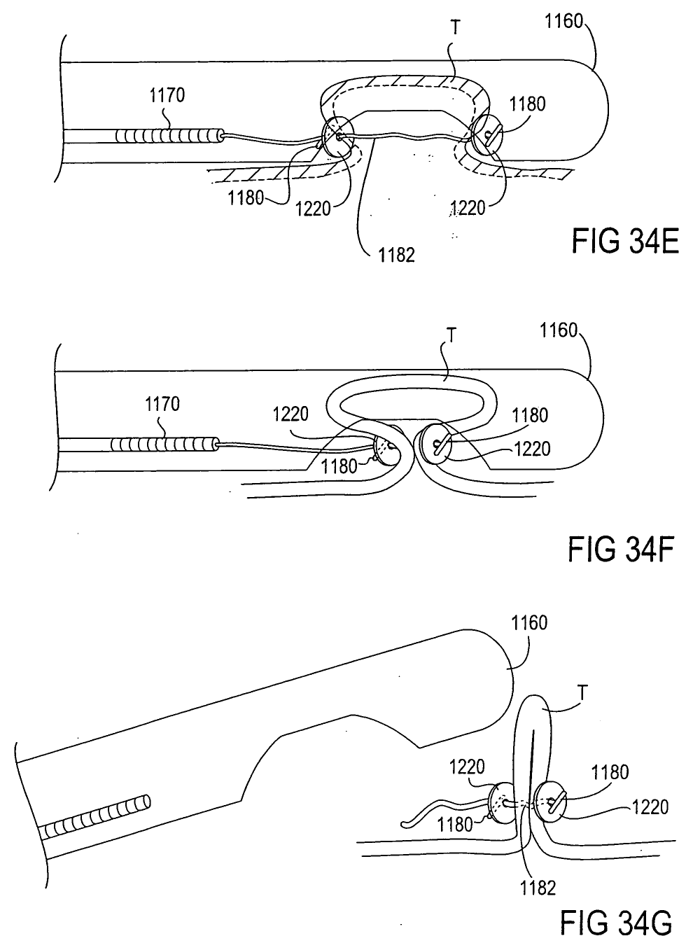

FIGS. 34A-34G illustrate an additional embodiment of a plication system of the present invention. This embodiment is similar to FIGS. 26A-26G, however in this embodiment pledgets 1220 are disposed within the suction cap 1160 on opposite sides of port 1162, as shown in FIG. 34A. It may be appreciated that the pledgets 1220 may have any shape or size and are used to increase the surface area of the stress applied to opposite sides of a tissue bulb during plication. As shown in FIG. 34B, tissue T is drawn into port 1162, between the pledgets 1220, forming a tissue bulb. A needle 1168 is advanced through the pledgets 1220 and the tissue T as shown in FIG. 34C. Anchors 1180 are released on opposite sides of the pledgets 1220, as shown in FIGS. 34D-34E; the anchors 1180 connected by a suture 1182. The suture 1182 is then cinched, as shown in FIG. 34F, to draw the anchors 1180 and pledgets 1220 together at the base of the tissue bulb, thereby securing and maintaining the plicated tissue T. The suction cap 1160 is then removed leaving the tissue T plicated by the anchors 1180 and pledgets 1220, as shown in FIG. 34G.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. An endoluminal apparatus comprising:
an elongated main body having a proximal section, a distal section configured for advancement through a body lumen, and a plurality of lumens extending therethrough, wherein
the proximal section is deflectable,
the distal section is steerable independently of the proximal section, and
at least one of the sections is comprised of a plurality of adjacent links which are capable of being transitioned from a flexible state to a rigidized state.

2. The apparatus of claim 1 wherein the distal section comprises an atraumatic tip having a plurality of openings corresponding to the plurality of lumens.

3. The apparatus of claim 1 wherein the proximal and/or distal sections are independently lockable in the rigidized state.

4. The apparatus of claim 1 wherein the proximal section is comprised of the plurality of adjacent links, wherein each link is configured to allow rotation and the links are arranged so that the proximal section is deflectable in a plurality of planes.

5. The apparatus of claim 1 wherein the distal section is comprised of the plurality of adjacent links, wherein each link is configured to allow rotation and the links are arranged so that the proximal section is deflectable in a plurality of planes.

6. The apparatus of claim 1 wherein the plurality of adjacent links comprise links which are nestable relative to each other.

7. The apparatus of claim 1 wherein the distal section is steerable within any axial plane in a 360 degree circumference around the proximal section.

8. The apparatus of claim 7 wherein the distal section is steerable within only one of the axial planes in a 360 degree circumference around the second section.

9. The apparatus of claim 1 further comprising at least one pullwire routed through the elongated main body, wherein compression of the plurality of adjacent links against one another relative to the pullwire transitions at least one of the sections to the rigidized state.

10. The apparatus of claim 1 further comprising at least one liner extending along a length of the elongated main body.

11. The apparatus of claim 10 wherein the at least one liner is torquable.

12. The apparatus of claim 1 further comprising a plurality of liners each disposed within a corresponding lumen through the elongated main body.

13. The apparatus of claim 12 wherein the plurality of liners are hydrophilically coated.

14. The apparatus of claim 1 wherein at least one of the lumens is adapted for irrigation and/or suction.

15. The apparatus of claim 1 wherein at least one of the lumens terminate in a side opening along the proximal or distal section.

16. The apparatus of claim 1 wherein at least one of the lumens is sized for passage of an endoscope.

17. The apparatus of claim 1 further comprising an endoscope for passage through at least one of the lumens.

18. The apparatus of claim 17 wherein a distal end of the endoscope is positionable in an off-axis position relative to the elongated main body such that a region of interest distal to the elongated main body is viewed at an angle via the endoscope.

19. The apparatus of claim 1 wherein at least one of the sections is comprised of a flexible or semi-flexible tube.

20. The apparatus of claim 1, wherein the proximal section is comprised of a continuous length of material.

21. The apparatus of claim 1 further comprising a Y-port located along the proximal section, wherein the Y-port is in communication with at least one of the lumens extending through the elongated main body.

22. An endoluminal apparatus comprising:
an elongated main body having a proximal section, a distal section configured for advancement through a body lumen, and a plurality of lumens extending therethrough, wherein
the proximal section is deflectable,
the distal section is steerable independently of the proximal section, and
each section is comprised of a plurality of adjacent links such that the distal section is steerable when the proximal section is transitioned from a flexible state to a rigidized state.

23. The apparatus of claim 22 wherein the distal section comprises an atraumatic tip having a plurality of openings corresponding to the plurality of lumens.

24. The apparatus of claim 22 wherein the proximal and/or distal sections are independently lockable in the rigidized state.

25. The apparatus of claim 22 wherein each link is configured to allow rotation and the links are arranged so that each section is deflectable in a plurality of planes.

26. The apparatus of claim 22 wherein the plurality of adjacent links comprise links which are nestable relative to each other.

27. The apparatus of claim 22 wherein the distal section is steerable within any axial plane in a 360 degree circumference around the proximal section.

28. The apparatus of claim 22 further comprising at least one pullwire routed through the elongated main body, wherein compression of the plurality of adjacent links against one another relative to the pullwire transitions at least one of the sections to the rigidized state.

29. The apparatus of claim 22 further comprising at least one liner extending along a length of the elongated main body.

30. The apparatus of claim 29 wherein the at least one liner is torquable.

31. The apparatus of claim 22 further comprising a plurality of liners each disposed within a corresponding lumen through the elongated main body.

32. The apparatus of claim 31 wherein the plurality of liners are hydrophilically coated.

33. The apparatus of claim 22 wherein at least one of the lumens is adapted for irrigation and/or suction.

34. The apparatus of claim 22 wherein at least one of the lumens terminate in a side opening along the proximal or distal section.

35. The apparatus of claim 22 wherein at least one of the lumens is sized for passage of an endoscope.

36. The apparatus of claim 22 further comprising an endoscope for passage through at least one of the lumens.

37. The apparatus of claim 36 wherein a distal end of the endoscope is positionable in an off-axis position relative to the elongated main body such that a region of interest distal to the elongated main body is viewed at an angle via the endoscope.

38. The apparatus of claim 22 wherein at least one of the sections is comprised of a flexible or semi-flexible tube.

39. The apparatus of claim 22 wherein the proximal section is comprised of a continuous length of material.

40. The apparatus of claim 22 further comprising a Y-port located along the proximal section, wherein the Y-port is in communication with at least one of the lumens extending through the elongated main body.
